(19) United States
(12) Patent Application Publication
(10) Pub. No.: US 2021/0113739 A1
(43) Pub. Date: Apr. 22, 2021

(54) TOPOLOGICAL ADHESION OF MATERIALS

(71) Applicant: President and Fellows of Harvard College, Cambridge, MA (US)

(72) Inventors: Jiawei YANG, Malden, MA (US); Ruobing BAI, Somerville, MA (US); Zhigang SUO, Lexington, MA (US)

(21) Appl. No.: 17/048,803

(22) PCT Filed: Mar. 19, 2019
(86) PCT No.: PCT/US19/22890

§ 371 Date: Oct. 19, 2020

Related U.S. Application Data
(60) Provisional application No. 62/660,348, filed on Apr. 20, 2018.

(51) Int. Cl. A61L 27/48; A61L 27/52; A61L 27/20

(52) U.S. Cl. CPC A61L 27/48; A61L 27/20; A61L 27/52

(57) ABSTRACT

A composite material is described, including a first material comprising a first polymeric network; a second material comprising a second polymeric network; and an adhesion polymeric network comprising a plurality of adhesion polymer chains joined together by a bonding force and interwoven with the first and second polymeric networks to adhere the first and second materials together, where the adhesion polymeric network is not covalently bonded with the first or second material. Methods of making such composite material are also described.

### CROSS-REFERENCES TO RELATED APPLICATIONS

This application claims the benefit and priority of U.S. Provisional Patent Application No. 62/660,348, filed on Apr. 20, 2018, the contents of which are hereby incorporated by reference in their entirety.

### STATEMENT REGARDING FEDERALLY SPONSORED RESEARCH OR DEVELOPMENT

This invention was made with government support from NSF MRSEC (Grant No. DMR-14-20570). The U.S. Government has certain rights in the invention.

### INCORPORATION BY REFERENCE

All patents, patent applications and publications cited herein are hereby incorporated by reference in their entirety in order to more fully describe the state of the art as known to those skilled therein as of the date of the invention described herein.

### FIELD OF THE INVENTION

The invention relates generally to the field of materials. More particularly, the invention relates to polymeric materials useful as adhesion materials.

### BACKGROUND

Existing and emerging medical practices have posed a persistent, fundamental challenge: creating strong adhesion between wet materials (e.g., living tissues and synthetic hydrogels) under physiological conditions. Applications include tissue repair, wound dressing, and drug delivery. Also under intense development are implantable devices for energy harvesting, neural stimulation and recording, sensing, and actuation. Existing adhesives are weak, or toxic, or incompatible with wet and soft surfaces, or restricted to specific functional groups from the wet materials. For example, cyanoacrylate is a strong adhesive, but is cytotoxic, and forms a glassy phase and hardens the interface. Nanoparticles, bridging polymers, fibrin, and polyethylene glycol gels are facile, but the adhesion energy is low (1-10 Jm⁻²), due to either weak bonds or fragile materials. A family of recently developed adhesives achieve adhesion energy of 1,000 Jm⁻², but the adhesion relies on functional groups from the tissues and hydrogels, and cannot bond hydrogels without suitable functional groups. Therefore, there remains a need for developing materials capable of achieving strong adhesion between materials, e.g., wet materials.

### SUMMARY

In one aspect, an adhesion polymeric network is used to achieve strong, topological adhesion between two materials, where the adhesion polymeric network forms no covalent bonds with the two materials. The adhesion polymeric network interweaves into the two materials and/or the polymeric networks within the two materials to achieve the topological adhesion. In certain embodiments, in response to a trigger, the polymer chains form a network in topological entanglement with the existing polymer networks of the two materials, stitching them together like a suture at the molecular scale. In some embodiments, the adhesion polymeric network is stretchable and/or flexible. In some embodiments, the adhesion polymeric network is mechanically compatible with both of the two materials (also referred to adherends) and does not restrict deformation of both of the two materials (also referred to adherends).

In one aspect, a composite material is described, including:

a first material including a first polymeric network;

a second material including a second polymeric network; and

an adhesion polymeric network including a plurality of adhesion polymer chains joined together by a bonding force and interwoven with the first and second polymeric networks to adhere the first and second materials together,
where the adhesion polymeric network is not covalently bonded to the first or second material or where the adhesion polymeric network is not covalently bonded to the first or second polymeric network.

In any one or more of the embodiments described herein, the first and/or second polymeric network is cross-linked.

In any one or more of the embodiments described herein, the first and/or second material is a dry material or a wet material including a solvent.

In any one or more of the embodiments described herein, the solvent is water.

In any one or more of the embodiments described herein, the solvent is an organic solvent.

In any one or more of the embodiments described herein, each of the first and second materials is independently selected from the group consisting of a hydrogel, a tissue, and an elastomer.

In any one or more of the embodiments described herein, the first and second polymeric networks each independently comprise one or more polymers selected from the group consisting of poly(hydroxyethylmethacrylate) (PHEMA), poly(acrylamide) (PAAM), poly(dimethylacrylamide) (PDMA), poly(N-isopropylacrylamide) (PNIPAM), sodium polyacrylate (NaPAA), [2-(Acryloyloxy)ethyl] trimethylammonium chloride (PDMAEA), polyacrylamide, alginate, and a combination thereof.

In any one or more of the embodiments described herein, the adhesion polymer chains include one or more bio-compatible polymers.

In any one or more of the embodiments described herein, each of the adhesion polymer chains is independently selected from the group consisting of poly(4-aminestyrene), chitosan, alginate, cellulose, poly(N-isopropylacrylamide), polymers containing silane groups and/or catechol groups, a copolymer thereof, a terpolymer thereof, and a block copolymer thereof.

In any one or more of the embodiments described herein, the bonding force results from a bond or interaction selected from the group consisting of hydrogen bond, ionic bond, van der Waals interaction, covalent bond, π-π stacking, cation-π interaction, host-guest interaction, and a combination thereof.

In any one or more of the embodiments described herein, the bonding force results from a bond or interaction which is permanent, transient, or reversible.

In any one or more of the embodiments described herein, the bond or interaction is reversible.

In any one or more of the embodiments described herein, the bond is a hydrogen bond, a covalent bond or an ionic bond.

In any one or more of the embodiments described herein, each of the adhesion polymer chains is independently selected from the group consisting of poly(4-aminestyrene), chitosan, alginate, cellulose, poly(N-isopropylacrylamide), polymers containing silane groups and/or catechol groups, a copolymer thereof, a terpolymer thereof, and a block copolymer thereof.

In any one or more of the embodiments described herein, the bonding force results from a bond or interaction which is formed in response to a stimulus.

In any one or more of the embodiments described herein, the stimulus is selected from the group consisting of pH, salt, temperature, light, and a combination thereof.

In any one or more of the embodiments described herein, the first and second materials are adhered with an adhesion energy of more than about 10, 50, 100, 200, 300, 500, 600, or 1000 Jm⁻².

In another aspect, a method of making a composite material is described, including:

providing a first material including a first polymeric network and a second material comprising a second polymeric network;

interweaving a plurality of adhesion polymer chains into the first and second polymeric networks;

joining two or more of the adhesion polymer chains together by a bonding force to form an adhesion polymeric network interwoven with the first and second polymeric networks to adhere the first and second materials together to form a composite material,

where the adhesion polymeric network is not covalently bonded to the first or second material; or where the adhesion polymeric network is not covalently bonded to the first or second polymeric network.

In any one or more of the embodiments described herein, the first and/or second material is a dry material or a wet material.

In any one or more of the embodiments described herein, the first and/or second material is a wet material comprising a solvent.

In any one or more of the embodiments described herein, the solvent is water or an organic solvent.

In any one or more of the embodiments described herein, each of the first and second materials is independently selected from the group consisting of a hydrogel, a tissue, and an elastomer.

In any one or more of the embodiments described herein, the first and second polymeric networks each independently comprises one or more polymers selected from the group consisting of poly(hydroxyethylmethacrylate) (PHEMA), poly(acrylamide) (PAAM), poly(dimethylacrylamide) (PDMA), poly(N-isopropylacrylamide) (PNIPAM), sodium polyacrylate (NaPAA), [2-(Acryloyloxy)ethyl] trimethylammonium chloride (PDMAEA), polyacrylamide, alginate, and a combination thereof.

In any one or more of the embodiments described herein, each of the adhesion polymer chains is independently selected from the group consisting of poly(4-aminestyrene), chitosan, alginate, cellulose, polymers containing silane groups and/or catechol groups, a copolymer thereof, a terpolymer thereof, and a block copolymer thereof.

In any one or more of the embodiments described herein, interweaving a plurality of adhesion polymer chains into the first and second polymeric networks includes contacting the first and second polymeric networks with a solution or a dispersion of the adhesion polymer chains in a solvent.

In any one or more of the embodiments described herein, the solvent is water or an organic solvent.

In any one or more of the embodiments described herein, joining two or more of the adhesion polymer chains together by a bonding force includes forming hydrogen bonds between the adhesion polymer chains, forming ionic bonds between the adhesion polymer chains, forming van der Waals interaction between the adhesion polymer chains, forming covalent bonds between the adhesion polymer chains, forming π-π stacking between the adhesion polymer chains, forming cation-π interaction between the adhesion polymer chains, forming host-guest interaction between the adhesion polymer chains, or a combination thereof.

In any one or more of the embodiments described herein, joining two or more of the adhesion polymer chains together by a bonding force includes forming hydrogen bonds, covalent bonds or ionic bonds between the adhesion polymer chains.

In any one or more of the embodiments described herein, joining two or more of the adhesion polymer chains together includes applying a stimulus to join the two or more of the adhesion polymer chains by the bonding force.

In any one or more of the embodiments described herein, the stimulus is selected from the group consisting of pH, salt, temperature, light, and a combination thereof.

In any one or more of the embodiments described herein, applying the stimulus includes changing the pH value of an aqueous solution or dispersion comprising the adhesion polymer chains, contacting the adhesion polymer chains with a plurality of positive or negative ions, subjecting the adhesion polymer chains to heating, subjecting the adhesion polymer chains to light, or a combination thereof.

In any one or more of the embodiments described herein, interweaving a plurality of adhesion polymer chains into the first and second polymeric networks incudes contacting the first and second polymeric networks with a solution or dispersion including the adhesion polymer chains in a solvent.

In any one or more of the embodiments described herein, the solution or dispersion is aqueous; and joining two or more of the adhesion polymer chains together to form the adhesion polymeric network includes changing the pH value of the solution or dispersion from a first value to a second value, wherein hydrogen bonds between the adhesion polymer chains form when pH is at the second value but do not form when pH is at first value.

In any one or more of the embodiments described herein, the first and/or second material is a wet material including water.

In any one or more of the embodiments described herein, the first and/or second material has a pH value the same or substantially the same as the second value.

In any one or more of the embodiments described herein, the method further includes changing the pH value of the solution or dispersion from the second value to the first value to break the hydrogen bonds.

In any one or more of the embodiments described herein, the adhesion polymer chain is poly(4-aminestyrene) and the first value is less than about 4.5 and the second value is more than about 4.5.

In any one or more of the embodiments described herein, the adhesion polymer chain is chitosan and the first value is less than about 6.5 and the second value is more than about 6.5.

In any one or more of the embodiments described herein, the adhesion polymer chain is alginate and the first value is more than about 3.5 and the second value is less than about 3.5.

In any one or more of the embodiments described herein, the adhesion polymer chain is cellulose and the first value is more than about 13 and the second value is less than about 13.

In any one or more of the embodiments described herein, the adhesion polymer chains include negative ions and joining two or more of the adhesion polymer chains together includes contacting the adhesion polymer chains with a plurality of positive ions and forming ionic bonds between the adhesion polymer chains to form the adhesion polymeric network.

In any one or more of the embodiments described herein, the positive ion includes Be²⁺, Mg²⁺, Ca²⁺, Sr²⁺, Ba²⁺, Mn²⁺, Fe²⁺, Cu²⁺, Ni²⁺, Zn²⁺, Al³⁺, Ga³⁺, Fe³⁺, or a combination thereof.

In any one or more of the embodiments described herein, the adhesion polymer chains include O⁻ or COO⁻.

In any one or more of the embodiments described herein, the adhesion polymer chains include a polymer selected from the group consisting of alginate, poly(acrylic acid), and copolymers consisting COO⁻.

In any one or more of the embodiments described herein, the method further includes contacting the adhesion polymeric network with a plurality of ion-exchanging positive ions to replace the positive ions to break the ionic bonds between the adhesion polymer chains.

In any one or more of the embodiments described herein, the ion-exchanging positive ion is H⁺, NH₄⁺, Li⁺, Na⁺, K⁺, Cs⁺, Rb⁺, or a combination thereof.

In any one or more of the embodiments described herein, the adhesion polymer chains comprise positive ions and joining two or more of the adhesion polymer chains together comprises contacting the adhesion polymer chains with a plurality of negative ions and forming ionic bonds between the adhesion polymer chains to form the adhesion polymeric network.

In any one or more of the embodiments described herein, the negative ions include Cl⁻, OH⁻, F⁻, CO₃²⁻, SO₄²⁻, HPO₄²⁻, PO₄³⁻, or a combination thereof.

In any one or more of the embodiments described herein, the adhesion polymer chains comprise —NH₃⁺, —NH₂R⁺, —NHR₂⁺, or a combination thereof, wherein each occurrence of R is alkyl, cycloalkyl, alkenyl, cycloallyl, or two R groups and the nitrogen atom taken together form a saturated, partially saturated, or unsaturated heterocycle.

In any one or more of the embodiments described herein, the adhesion polymer chains include a polymer selected from the group consisting of chitosan and poly(4-aminestyrene).

In any one or more of the embodiments described herein, the method further includes contacting the adhesion polymeric network with a plurality of ion-exchanging negative ions to replace the negative ions to break the ionic bonds between the adhesion polymer chains.

In any one or more of the embodiments described herein, the ion-exchanging negative ion is OH⁻, F⁻, Cl⁻, Br⁻, I⁻, NO₃⁻, or a combination thereof.

In any one or more of the embodiments described herein, joining two or more of the adhesion polymer chains together includes heating the adhesion polymer chains to cross-link the adhesion polymer chains to form the adhesion polymeric network.

In any one or more of the embodiments described herein, the adhesion polymer chains are in contact with one or more nanoparticles capable of generating heat upon exposure to light; and heating the adhesion polymer chains including subjecting the nanoparticles to light.

In any one or more of the embodiments described herein, the nanoparticles are gold nanoparticles.

In any one or more of the embodiments described herein, the adhesion polymer chain is a thermo-responsive polymer.

In any one or more of the embodiments described herein, the thermo-responsive polymer is poly(N-isopropylacrylamide) (PNIPAM).

In any one or more of the embodiments described herein, the method further includes cooling the adhesion polymeric network to break the cross-links between the adhesion polymer chains.

In any one or more of the embodiments described herein, joining two or more of the adhesion polymer chains together by a bonding force includes forming covalent bonds between the adhesion polymer chains.

Any aspect or embodiment disclosed herein may be combined with another aspect or embodiment disclosed herein. The combination of one or more embodiments described herein with other one or more embodiments described herein is expressly contemplated.

As used herein, the term “polymer chain” refers to a molecular chain formed by multiple repeat units that are covalently linked to one another, where a portion of the polymer chain (e.g., less than about 10%, 5%, 3%, or 1% of the monomer repeating units in the polymer chain) may optionally be branched or crosslinked. As used herein, “polymeric network” refers to a plurality of polymer chains optionally branched or crosslinked at certain monomer repeating unit (e.g., about or less than about 10%, 5%, 3%, or 1% of the monomer repeating units in the polymer chain). As used herein, the “adhesion polymeric chain” refers to a polymer which is capable of interweaving into the first and second polymeric networks of the first and second materials, respectively, but does not form any covalent bond with the first and second polymeric networks or the first and second materials. As used herein, the “adhesion polymeric network” refers to a polymeric network formed by joining a plurality of the adhesion polymer chains together by a bonding force, where the adhesion polymeric network is interwoven with the first and second polymeric networks to adhere the first and second materials together. In some embodiments, the adhesion polymeric network is stable, stretchable and/or flexible.

As used herein, the term “polymer” includes, but is not limited to, the homopolymer, copolymer, terpolymer, and block copolymer, random copolymers and terpolymers of that polymer. As used herein, copolymer refers to a polymer derived from two monomeric species; similarly, a terpolymer refers to a polymer derived from three monomeric species. Block copolymers include, but are not limited to, block, graft, dendrimer, and star polymers. The polymer also includes various morphologies, including, but not limited to, linear polymer, branched polymer, crosslinked polymer, and dendrimer systems. As an example, polyacrylamide polymer refers to any polymer including polyacrylamide, e.g., a homopolymer, copolymer, terpolymer, block copolymer, or terpolymer of polyacrylamide. Polyacrylamide can be a linear polymer, branched polymer, crosslinked polymer, or a dendrimer of polyacrylamide.

Unless otherwise defined, used, or characterized herein, terms that are used herein (including technical and scientific terms) are to be interpreted as having a meaning that is consistent with their accepted meaning in the context of the relevant art and are not to be interpreted in an idealized or overly formal sense unless expressly so defined herein.

Although the terms, first, second, third, etc., may be used herein to describe various elements, these elements are not to be limited by these terms. These terms are simply used to distinguish one element from another. Thus, a first element, discussed below, could be termed a second element without departing from the teachings of the exemplary embodiments. Spatially relative terms, such as “above,” “below,” “left,” “right,” “in front,” “behind,” and the like, may be used herein for ease of description to describe the relationship of one element to another element, as illustrated in the figures. It will be understood that the spatially relative terms, as well as the illustrated configurations, are intended to encompass different orientations of the apparatus in use or operation in addition to the orientations described herein and depicted in the figures. For example, if the apparatus in the figures is turned over, elements described as “below” or “beneath” other elements or features would then be oriented “above” the other elements or features. Thus, the exemplary term, “above,” may encompass both an orientation of above and below. The apparatus may be otherwise oriented (e.g., rotated 90 degrees or at other orientations) and the spatially relative descriptors used herein interpreted accordingly. Further still, in this disclosure, when an element is referred to as being “linked to,” “on,” “connected to,” “coupled to,” “in contact with,” etc., another element, it may be directly linked to, on, connected to, coupled to, or in contact with the other element or intervening elements may be present unless otherwise specified.

The terminology used herein is for the purpose of describing particular embodiments and is not intended to be limiting of exemplary embodiments. As used herein, singular forms, such as “a” and “an,” are intended to include the plural forms as well, unless the context indicates otherwise. Additionally, the terms “includes,” “including,” “comprises,” and “comprising” specify the presence of the stated elements or steps but do not preclude the presence or addition of one or more other elements or steps.

### DESCRIPTION OF THE DRAWINGS

The invention is described with reference to the following figures, which are presented for the purpose of illustration only and are not intended to be limiting. In the Drawings:

FIGS. 1A-1E demonstrate the principle of pH-triggered topological adhesion, according to one or more embodiments described herein. FIG. 1A shows chitosan chains dissolving in water at pH=5. FIG. 1B shows chitosan chains forming a network in water at pH=7. FIG. 1C shows an aqueous solution of chitosan of pH=5 placed between two hydrogels of pH=7.

FIG. 1D illustrates one aspect of the application using the example of chitosan chains diffused into the two hydrogels and forming a network. The chitosan network is topologically entangled with the network of a hydrogel on either side, and stiches the two hydrogels together.

FIG. 1E shows confocal microscopic images of a chitosan solution placed between two pieces of polyacrylamide hydrogels. A sequence of confocal microscopic images show that the chitosan chains diffuse away from the interface in the first hour, gradually diffuse less for the next few hours, and cease to diffuse at 24 hours. The remaining chitosan chains near the interface form a network, which prohibits chitosan chains to diffuse away. The scale bar is 300 μm.

FIG. 1F shows the adhesion of various hydrogels, including neutral hydrogels (polyacrylamide (PAAM), poly(dimethylacrylamide) (PDMA), poly(hydroxyethylmethacrylate) (PHEMA), poly(N-isopropylacrylamide) (PNIPAM), a negatively charged hydrogel (sodium polyacrylate (NaPAA), a positively charged hydrogel ([2-(Acryloyloxy)ethyl] trimethylammonium chloride (PDMAEA), and a tough hydrogel (polyacrylamide/alginate (alg-PAAM), according to one or more embodiments described herein. The adhesion energy increases with the bulk toughness of hydrogel. The data represents the mean and standard deviation of 4-6 experimental results.

FIG. 1G shows the adhesion energy of the stitching of PAAM hydrogel to various porcine tissues: liver, heart, artery, and skin using chitosan chains. The data represents the mean and standard deviation of 4-6 experimental results.

FIGS. 2A-2E demonstrate the adhesion energy as a function of several variables, according to one or more embodiments described herein. FIG. 2A shows the schematics of a bonding procedure, including: spreading an aqueous solution of chitosan on the surface of one PAAM hydrogel; placing the other PAAM hydrogel on top, and compressing the two pieces of hydrogels with strain d/L. FIG. 2B illustrates the adhesion energy varying with the thickness of the chitosan solution and the compressive strain. FIG. 2C illustrates the adhesion energy's change over time. FIG. 2D illustrates that the adhesion energy increases with the concentration of chitosan solution. FIG. 2E illustrates that the adhesion energy increases with the molecular weight of chitosan chains. All the data represents the mean and standard deviation of 4-6 experimental results.

FIGS. 3A-3G demonstrate the adhesion in full range of pH using various polymers as examples, according to one or more embodiments described herein. FIG. 3A shows that strong adhesion in all pH levels can be achieved by selecting four species of stitching polymers: cellulose, chitosan, poly(4-aminestyrene) (PAS), and alginate. The range in which these stitching polymers form network covers all pH levels. The adhesion energy depends on pH when two pieces of PAAM hydrogels are bonded with each species of stitching polymer chains: alginate (FIG. 3B), PAS (FIG. 3C), chitosan (FIG. 3D), and cellulose (FIG. 3E).

FIGS. 3F-3G demonstrate on-demand deactivation of adhesion, according to one or more embodiments described herein. Two pieces of PAAM hydrogels were bonded with chitosan, with one hydrogel gluing on the top rigid plate and the other hanging a weight. In FIG. 3F, when water was dripped at the bonding front, the hydrogels remained bonded. In FIG. 3G, when hydrochloric acid was dripped at the bonding front, the hydrogels debonded. The scale bar is 2 cm.

FIG. 4 demonstrates crack speed as a function of energy release rate, according to one or more embodiments described herein.

FIG. 5A demonstrates that an aqueous solution of chitosan of pH=5 was spread at the interface of two hydrogels of pH=5. FIG. 5B demonstrates that the chitosan chains diffused into both hydrogels and, because chitosan is soluble at pH=5, the chitosan chains diffused across the entire hydrogels and did not form a network. FIG. 5C shows a sequence of confocal images showing that the chitosan chains diffused away from the interface. The chitosan chains kept diffusing away even after 23 hours. The scale bar is 300 μm.

FIGS. 6A-6E demonstrate a T-peeling test, according to one or more embodiments described herein. FIG. 6A shows the schematics of a hydrogel bilayer. FIG. 6B shows the schematics of the T-peeling test. FIG. 6C shows a photo of the T-peeling test. FIG. 6D shows a photo of the T-peeling test conducted in a humidity chamber. FIG. 6E shows the representative force-displacement curve of the T-peeling test. The adhesion energy is calculated as two times the steady-state force at the plateau divided by the width of the bilayer. The scale bar in FIGS. 6C-6D is 1 cm.

FIGS. 7A-7B demonstrate a visual comparison of chitosan precipitation, according to one or more embodiments described herein. FIG. 7A shows that when a chitosan solution (pH=5) was dripped with NaOH (1 M), chitosan chains precipitated out and the solution became turbid. FIG. 7B illustrates that when chitosan chains bonded two pieces of PAAM hydrogels with pH>7, the interface remains optically transparent. The scale bar is 5 mm.

FIG. 8 illustrates the adhesion kinetics for PAAM hydrogels bonded with PAS, according to one or more embodiments described herein. The adhesion energy builds up to ˜300 Jm⁻² within 15 minutes after bonding, and reaches an equilibrium value of ˜400 Jm⁻² about 10 hours.

FIG. 9 is a demonstration of tissue adhesives used in extremely acidic environment, according to one or more embodiments described herein. A piece of gastric acid-treated porcine stomach tissue (pH˜1.5) was bonded with a PAAM hydrogel using cellulose. Under uniaxial tension, the hydrogel was stretched as large as 11 times its initial length without debonding. The scale bar is 3 cm.

### DETAILED DESCRIPTION

In one aspect, a composite material is described, including:

a first material including a first polymeric network;

a second material including a second polymeric network; and

an adhesion polymeric network including a plurality of adhesion polymer chains joined together by a bonding force and interwoven with the first and second polymeric networks to adhere the first and second materials together,

where the adhesion polymeric network is not covalently bonded to the first or second material; or where the adhesion polymeric network is not covalently bonded to the first or second polymeric network.

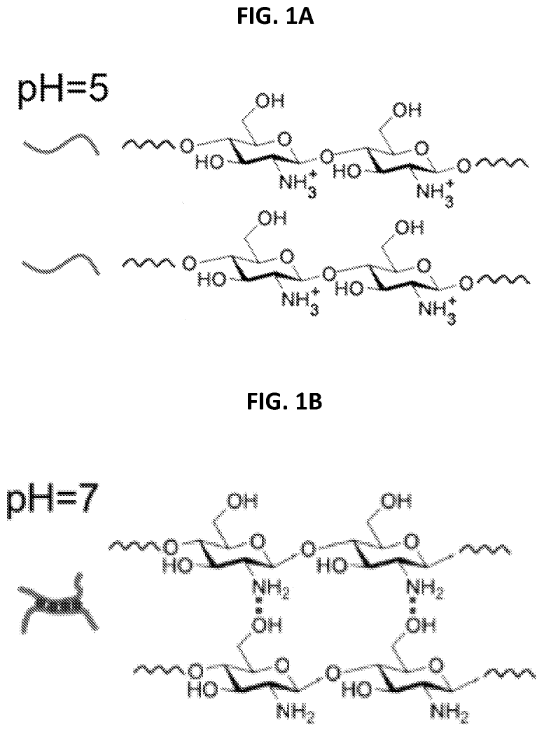

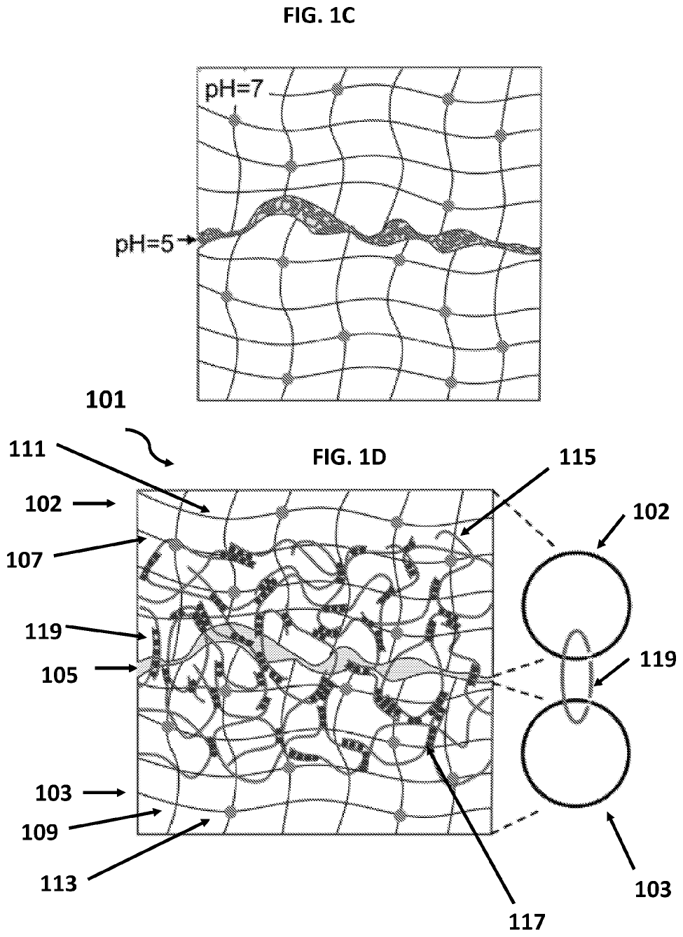

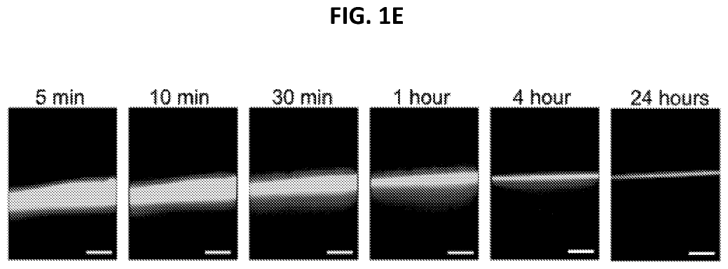

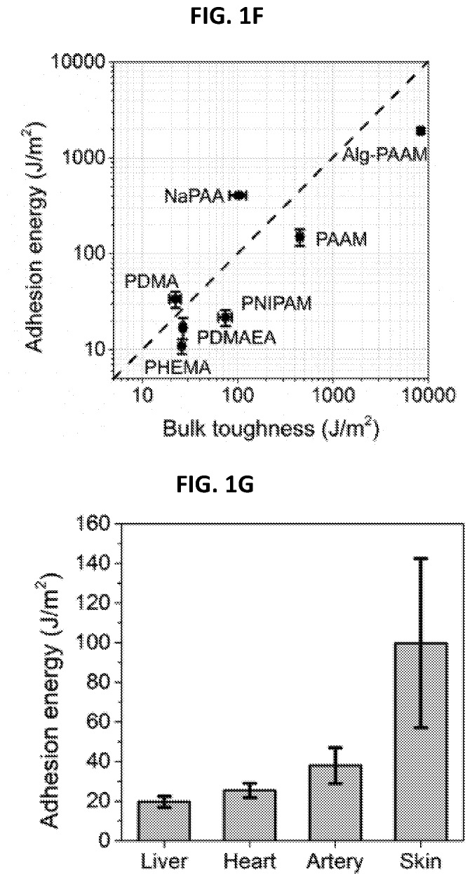

In some embodiments, the adhesion polymeric network is not covalently bonded to the first or second material. In other embodiments, the adhesion polymeric network is not covalently bonded to the first or second polymeric network. As used herein, the term “bonding force” refers to any forces of attraction which act between neighboring adhesion polymer chains to join them together to form a stable and/or stretchable adhesion polymeric network. The invention is now described with reference to FIG. 1D, which provides non-limiting examples of the aspects and/or embodiments disclosed herein. FIG. 1D shows a composite material 101, including a first material 102, a second material 103 and an adhesion polymeric network 119 topologically adhering the first and second materials 102 and 103 together. The adhesion polymeric network 119 resides partially in the interface region 105 between the first and second materials 102 and 103 and partially in the first and second materials 102 and 103. The first material 102 contains a first polymeric network 107, which may include a plurality of first polymer chain 111 cross-linked together to form the first polymeric network 107. Similarly, the second material 103 contains a second polymeric network 109, which may include a plurality of second polymer chain 113 cross-linked together to form the second polymeric network 109. As shown in the Figure, the adhesion polymeric network is not covalently bonded to the first or second polymeric network (or the first and second material).

In certain embodiments, the first and/or second material is a dry material. In other embodiments, the first and/or second material is a wet material including a solvent. Non-limiting examples of the solvent include water and an organic solvent, which includes, but is not limited to, ethanol, dichloromethane, THF, acetone, acetonitrile, toluene, and a combination thereof.

In certain embodiments, the first and second materials are each independently selected from the group consisting of a hydrogel, a tissue or an elastomer. Thus, the first and second materials 102 and 103 can both be hydrogels, both be tissues, both be elastomers, be a combination of a hydrogel and a tissue, be a combination of a hydrogel and an elastomer, or be a combination of an elastomer and a tissue. In some embodiments, the first and second polymeric networks 107 and 109 each independently include one or more polymers selected from the group consisting of poly(hydroxyethylmethacrylate) (PHEMA), poly(acrylamide) (PAAM), poly(dimethylacrylamide) (PDMA), poly(N-isopropylacrylamide) (PNIPAM), sodium polyacrylate (NaPAA), [2-(Acryloyloxy)ethyl] trimethylammonium chloride (PDMAEA), polyacrylamide, alginate, and a combination thereof.

In some embodiments, the adhesion polymeric network 119 includes a plurality of adhesion polymer chains 115. In these embodiments, the adhesion polymer chains 115 interweave into both the first polymeric network 107 and the second polymeric network 109 such that the adhesion polymer chains 115 and the first and second polymeric networks 107 and 109 become topologically entangled. In these embodiments, the plurality of the adhesion polymer chains 115 are joined together by a bonding force 117 to form the adhesion polymeric network 119. The adhesion polymeric network 119 is thus interwoven with the first and second polymer networks 107 and 109 and topologically adheres the first and second materials 102 and 103 together without any covalent bonds between the adhesion polymeric network and the first and second materials. The right-hand side of FIG. 1D shows a simplified schematic of the topological adhesion, where the first and second materials 102 and 103 are topologically “locked” or adhered together by the adhesion polymeric network 119.

In some embodiments, the adhesion polymer chain 115 includes a bio-compatible polymer. As used herein, a bio-compatible polymer refers to the polymer which is compatible with living tissue or a living system and is not toxic, injurious, physiologically reactive, and/or causing immunological rejection. In some embodiments, the adhesion polymer chain 115 is selected from the group consisting of poly(4-aminestyrene), chitosan, alginate, cellulose, poly(N-isopropylacrylamide), polymers containing any reactive functional groups, a copolymer thereof, a terpolymer thereof, and a block copolymer thereof. Non-limiting examples of polymers containing reactive functional groups include polymers including silane groups and/or catechol groups. Further examples of polymers containing silane groups and/or catechol groups are described in U.S. Provisional Application 62/635,882, the content of which is incorporated by reference.

In some embodiments, the adhesion polymer chain is also referred to as the stitching polymers and the adhesion polymeric network is also referred to as the stitching polymer network. In these embodiments, the topological adhesion is also referred to as topohesion for brevity and the stitching polymers is referred to as the topohesive. The stitching polymer network functions as a molecular suture.

Various bonds or interactions can result in the bonding force 117 joining the adhesion polymer chains 115 together to form the adhesion polymeric network 119. Non-limiting examples of the bonds or interactions include hydrogen bond, ionic bond, van der Waals interaction, covalent bond, π-π stacking, cation-π interaction, host-guest interaction, and a combination thereof. The bond or interaction can be permanent, transient, or reversible. In certain embodiments, the bond or interaction is reversible, i.e., the bond or interaction may be formed to join the adhesion polymer chains 115 together to form the adhesion polymeric network 119, and then broken to release the free the adhesion polymer chains 115. When the bond or interaction is reversible, the topological adhesion is “on-demand,” i.e., the first and second materials 102 and 103 may be adhered together and then dissociated from each other if needed.

Non-limiting examples of such reversible bonds or interactions include a hydrogen bond, an ionic bond, and a cross-linking bond between thermo-responsive polymers. A hydrogen bond is a partially electrostatic attraction between a hydrogen which is bound to a more electronegative atom such as nitrogen, oxygen, or fluorine (hydrogen bond donor), and another adjacent atom bearing a lone pair of electrons (hydrogen bond acceptor). In some embodiments, the adhesion polymer chain includes functional groups capable of serving as hydrogen bond donors to form hydrogen bond with the hydrogen bond acceptor atoms on an adjacent adhesion polymer chain. Non-limiting examples of such hydrogen bond donor functional groups include OH, COOH, and NH₂. In certain embodiments, the adhesion polymer chain is selected from the group consisting of poly(4-aminestyrene), chitosan, alginate, cellulose, polymers containing silane groups and catechol groups, a copolymer thereof, a terpolymer thereof, and a block copolymer thereof.

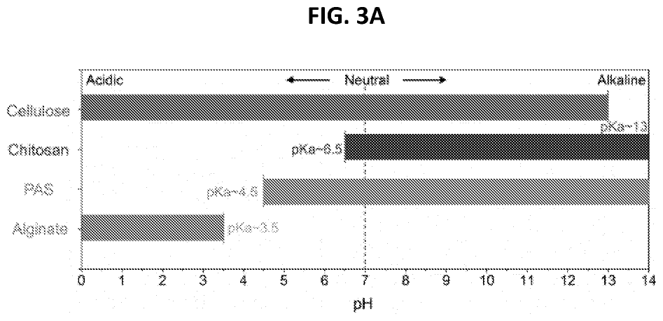

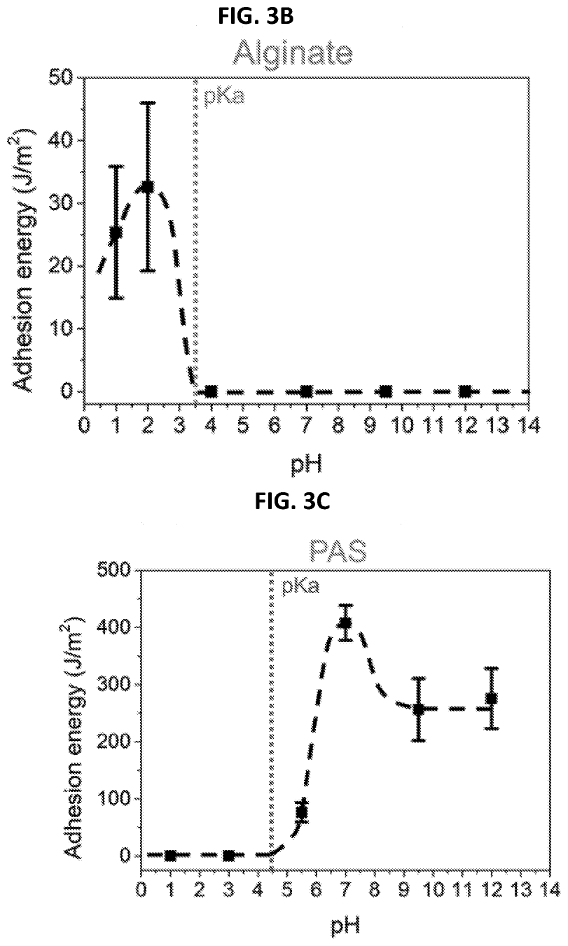

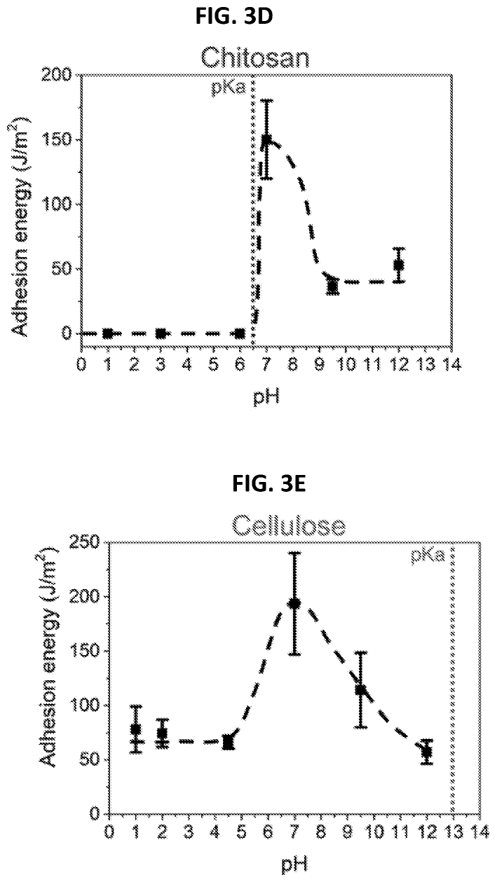

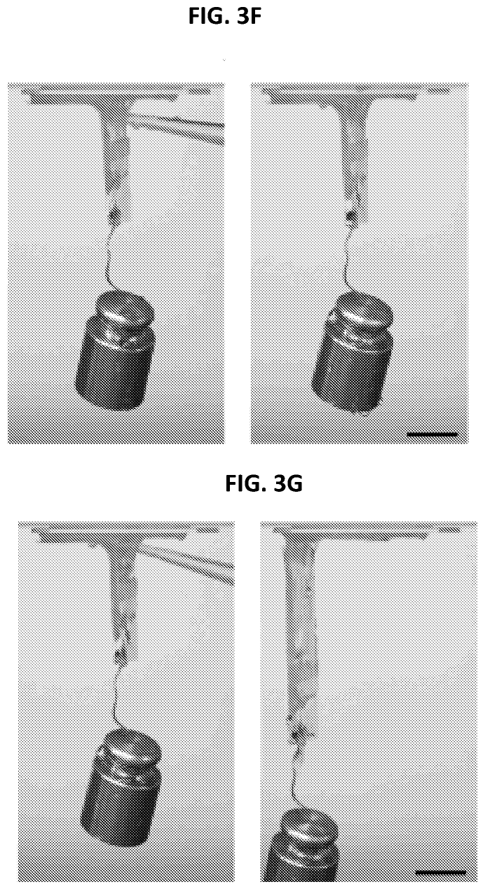

In some embodiments, the hydrogen bond joining the adhesion polymer chains to form the adhesion polymeric network is reversible. In certain embodiments, the hydrogen bond may be formed under a second value of pH, and be broken under a first value of pH. In some embodiments, the hydrogen bond is broken because the hydrogen bond acceptor atom no longer has the required electron lone pairs to interact with the hydrogen from the hydrogen bond donor. Non-limiting examples of these embodiments include the protonation of hydrogen bond acceptor —NH₂, —NHR, or —NR₂ to become the corresponding ammonium ion —NH₃⁺, —NH₂R⁺, or —NHR₂⁺, which no longer has the required electron lone pairs to interact with the hydrogen from the hydrogen bond donor. In certain embodiments, each occurrence of R is alkyl, cycloalkyl, alkenyl, cycloallyl, or two R groups and the nitrogen atom taken together form a saturated, partially saturated, or unsaturated heterocycle. Non-limiting examples of the adhesion polymer chains having —NH₂, —NHR, or —NR₂ as the hydrogen bond acceptor include poly(4-aminestyrene) (PAS) and chitosan. As shown in FIG. 3A, the hydrogen bonds joining the adhesion polymer chain chitosan or poly(4-aminestyrene) form when the pH is more than the pKa of its —NH₂ groups (i.e., pH>6.5 for chitosan and pH>4.5 for PAS), and break when the pH is less than the pKa of its —NH₂ groups (i.e., pH<6.5 for chitosan and pH<4.5 for PAS).

In other embodiments, the hydrogen bond is broken because the hydrogen bond donor no longer has the hydrogen for forming the hydrogen bond. This may be a result of a pH change. Non-limiting examples of these embodiments include the deprotonation of COOH or OH to form COO⁻ or O⁻. Non-limiting examples of the adhesion polymer chains having COOH or OH as the hydrogen bond donor include alginate and cellulose. As shown in FIG. 3A, the hydrogen bonds joining the adhesion polymer chain alginate and cellulose form when the pH is less than the pKa of its OH groups (i.e., pH<13 for cellulose and pH<3.5 for alginate), and break when the pH is more than the pKa of its —OH groups (i.e., pH>13 for cellulose and pH>3.5 for alginate).

In other embodiments, the adhesion polymer chains include positive or negative ions. In these embodiments, the bonds joining two or more of the adhesion polymer chains together are ionic bonds. Such ionic bonds may be formed upon a stimulus such as adding external positive ions (for adhesion polymer chains including negative ions) or negative ions (for adhesion polymer chains including positive ions).

In some embodiments, the adhesion polymer chains include O⁻ or COO⁻. Non-limiting examples of the external positive ions include Be²⁺, Mg²⁺, Ca²⁺, Sr²⁺, Ba²⁺, Mn²⁺, Fe²⁺, Cu²⁺, Ni²⁺, Zn²⁺, Al³⁺, Ga³⁺, Fe³⁺, or a combination thereof. In these embodiments, the adhesion polymer chains include a polymer selected from the group consisting of, alginate, poly(acrylic acid) and copolymers consisting COO⁻. In certain embodiments, the ionic bond joining the adhesion polymer chains to form the adhesion polymeric network is reversible, e.g., by the addition of ion-exchanging positive ions to replace the positive ions to break the ionic bonds between the adhesion polymer chains. Non-limiting examples of the ion-exchanging positive ions include H⁺, NH₄⁺, Na⁺, K⁺, Cs⁺, Rb⁺, and a combination thereof.

In other embodiments, the adhesion polymer chains include positive ion —NH₃⁺, —NH₂R⁺, —NHR₂⁺, or a combination thereof, where each occurrence of R is alkyl, cycloalkyl, alkenyl, cycloallyl, or two R groups and the nitrogen atom taken together form a saturated, partially saturated, or unsaturated heterocycle. Non-limiting examples of the external negative ions include Cl⁻, OH⁻, F⁻, CO₃²⁻, SO₄²⁻, HPO₄²⁻, PO₄³⁻, or a combination thereof. In these embodiments, the adhesion polymer chains include a polymer selected from the group consisting of poly(4-aminestyrene) (PAS) and chitosan. In certain embodiments, the ionic bond joining the adhesion polymer chains to form the adhesion polymeric network is reversible, e.g., by the addition of ion-exchanging negative ions to replace the positive ions to break the ionic bonds between the adhesion polymer chains. Non-limiting examples of the ion-exchanging negative ions include OH⁻, F⁻, Cl⁻, Br⁻, I⁻, NO₃⁻, or a combination thereof.

Thus, in certain embodiments, the bonding force results from a bond or interaction which is formed in response to a stimulus. Non-limiting examples of the stimuli include pH and salt as described above, as well as heat and light. In some embodiments, the adhesion polymer chain is a thermo-responsive polymer. Non-limiting examples of the thermo-responsive polymers include poly(N-isopropylacrylamide) (PNIPAM). In these embodiments, the adhesion polymer chains may be heated to cross-link the adhesion polymer chains to form the adhesion polymeric network.

In other embodiments, the thermo-responsive polymer is in contact with one or more nanoparticles capable of generating heat upon exposure to light. Non-limiting examples of such nanoparticles include gold nanoparticles. In these embodiments, when exposed to light, the nanoparticles generate heat, which causes the thermo-responsive polymer to cross-link to form the adhesion polymeric network.

In certain embodiments, the cross-linkage between the thermo-responsive polymer chains are reversible. In some specific embodiments, the adhesion polymeric network formed from the thermo-responsive polymer chains is cooled to break the cross-linkage between the thermo-responsive polymer chains.

The composite material described herein has the first and second materials topologically adhered together with high adhesion energy. In certain embodiments, the first and second materials are adhered with an adhesion energy of more than about 10, 50, 100, 200, 300, 500, 600, or 1000 Jm⁻².

In another aspect, a method of making a composite material is described, including:

providing a first material including a first polymeric network and a second material comprising a second polymeric network;

interweaving a plurality of adhesion polymer chains into the first and second polymeric networks;

joining two or more of the adhesion polymer chains together by a bonding force to form an adhesion polymeric network interwoven with the first and second polymeric networks to adhere the first and second materials together to form a composite material,

where the adhesion polymeric network is not covalently bonded to the first or second material.

As used herein, the term “interweave” or “interwoven” refers to the phenomena where two or more polymer chains or polymeric networks or a polymer chain and a polymeric network weave or become woven together.

In some embodiments, the first and/or second material is a dry material or a wet material. In certain specific embodiments, the first and/or second material is a wet material comprising a solvent. Non-limiting examples of the solvents include water and an organic solvent. As described herein, each of the first and second materials may be independently selected from the group consisting of a hydrogel a tissue, and an elastomer. In certain embodiments, the first and second polymeric networks each independently include one or more polymers selected from the group consisting of poly(hydroxyethylmethacrylate) (PHEMA), poly(acrylamide) (PAAM), poly(dimethylacrylamide) (PDMA), poly(N-isopropylacrylamide) (PNIPAM), sodium polyacrylate (NaPAA), [2-(Acryloyloxy)ethyl] trimethylammonium chloride (PDMAEA), polyacrylamide, alginate, and a combination thereof.

As described herein, each of the adhesion polymer chains is independently selected from the group consisting of poly(4-aminestyrene), chitosan, alginate, cellulose, poly(N-isopropylacrylamide), polymers containing silane groups and catechol groups, a copolymer thereof, a terpolymer thereof, and a block copolymer thereof.

In some embodiments, interweaving a plurality of adhesion polymer chains into the first and second polymeric networks include contacting the first and second polymeric networks with a solution or a dispersion of the adhesion polymer chains in a solvent. Non-limiting examples of solvents include water and an organic solvent. Non-limiting examples of organic solvents include ethanol, dichloromethane, THF, acetone, acetonitrile, toluene, and a combination thereof.

In some embodiments, the first and second materials are first provided and the adhesion polymer chains are provided, e.g., in a solution or dispersion, and placed in-between the first and second materials so that the adhesion polymer chains interweave into the first and second polymeric networks. In other embodiments, the first material and the adhesion polymer chains are first provided and in contact with each other. For instance, the adhesion polymer chains may be provided as a solution or dispersion to be in contact with the first material so that the adhesion polymer chains interweave into the first polymeric network. In these embodiments, the second polymeric network is then provided to be in contact with the adhesion polymer chains such that the adhesion polymer chains also interweave into the second polymeric network.

In some embodiments, joining two or more of the adhesion polymer chains together by a bonding force includes forming hydrogen bonds between the adhesion polymer chains, forming ionic bonds between the adhesion polymer chains, forming van der Waals interaction between the adhesion polymer chains, forming covalent bonds between the adhesion polymer chains, forming π-π stacking between the adhesion polymer chains, forming cation-π interaction between the adhesion polymer chains, forming host-guest interaction between the adhesion polymer chains, or a combination thereof.

In some embodiments, joining two or more of the adhesion polymer chains together includes applying a stimulus to join the two or more of the adhesion polymer chains. Non-limiting examples of the stimuli include pH, salt, temperature, light, and a combination thereof. In certain embodiments, the method includes changing the pH value of an aqueous solution or dispersion comprising the adhesion polymer chains, contacting the adhesion polymer chains with a plurality of positive or negative ions, subjecting the adhesion polymer chains to heating, subjecting the adhesion polymer chains to light, or a combination thereof.

In some specific embodiments, joining two or more of the adhesion polymer chains together by a bonding force includes forming hydrogen bonds between the adhesion polymer chains. In certain embodiments, interweaving a plurality of adhesion polymer chains into the first and second polymeric networks includes contacting the first and second polymeric networks with a solution or dispersion comprising the adhesion polymer chains in a solvent. In specific embodiments, the method includes changing the pH value of the solution or dispersion from a first value to a second value, wherein hydrogen bonds between the adhesion polymer chains form when pH is at the second value but do not form when pH is at the first value.

The first and/or second material may be a wet material including water. In some specific embodiments, the first and/or second material has a pH value the same or substantially the same as the second value. In some embodiments, the formation of the hydrogen bond is reversible and the method further includes changing the pH value of the solution or dispersion from the second value to the first value to break the hydrogen bonds.

In some specific embodiments, the adhesion polymer chain is poly(4-aminestyrene) and the first value is less than about 4.5 and the second value is more than about 4.5. In some specific embodiments, the adhesion polymer chain is chitosan and the first value is less than about 6.5 and the second value is more than about 6.5. In some specific embodiments, the adhesion polymer chain is alginate and the first value is more than about 3.5 and the second value is less than about 3.5. In some specific embodiments, the adhesion polymer chain is cellulose and the first value is more than about 13 and the second value is less than about 13.

In some specific embodiments, the adhesion polymer chains include negative ions and joining two or more of the adhesion polymer chains together includes contacting the adhesion polymer chains with a plurality of positive ions and forming ionic bonds between the adhesion polymer chains to form the adhesion polymeric network. As described above, non-limiting examples of the positive ions include Be²⁺, Mg²⁺, Ca²⁺, Sr²⁺, Ba²⁺, Mn²⁺, Fe²⁺, Cu²⁺, Ni²⁺, Zn²⁺, Al³⁺, Ga³⁺, Fe³⁺, or a combination thereof. In certain embodiments, the adhesion polymer chains include O⁻ or COO⁻. In certain embodiments, the adhesion polymer chains comprise a polymer selected from the group consisting of alginate, poly(acrylic acid), and copolymers consisting COO⁻. In certain embodiments, the formation of the ionic bonds joining the adhesion polymer chains is reversible and the method further includes contacting the adhesion polymeric network with a plurality of ion-exchanging positive ions to replace the positive ions to break the ionic bonds between the adhesion polymer chains. Non-limiting examples of the ion-exchanging positive ions include H⁺, NH₄⁺, Li⁺, Na⁺, K⁺, Cs⁺, Rb⁺, or a combination thereof.

In other specific embodiments, the adhesion polymer chains include positive ions and joining two or more of the adhesion polymer chains together include contacting the adhesion polymer chains with a plurality of negative ions and forming ionic bonds between the adhesion polymer chains to form the adhesion polymeric network. Non-limiting examples of the negative ions include Cl⁻, OH⁻, F⁻, CO₃²⁻, SO₄²⁻, HPO₄²⁻, PO₄³⁻, or a combination thereof. In certain specific embodiments, the adhesion polymer chains include —NH₃⁺, —NH₂R⁺, —NHR₂⁺, or a combination thereof, where each occurrence of R is alkyl, cycloalkyl, alkenyl, cycloallyl, or two R groups and the nitrogen atom taken together form a saturated, partially saturated, or unsaturated heterocycle. In these embodiments, the adhesion polymer chains include of chitosan and poly(4-aminestyrene). In certain embodiments, the formation of the ionic bonds joining the adhesion polymer chains is reversible and the method further includes contacting the adhesion polymeric network with a plurality of ion-exchanging negative ions to replace the negative ions to break the ionic bonds between the adhesion polymer chains. Non-limiting examples of the ion-exchanging negative ions include OH⁻, F⁻, Cl⁻, Br⁻, I⁻, NO₃⁻, or a combination thereof.

In still other specific embodiments, joining two or more of the adhesion polymer chains together includes heating the adhesion polymer chains to cross-link the adhesion polymer chains to form the adhesion polymeric network. In certain embodiments, the adhesion polymer chains are in contact with one or more nanoparticles capable of generating heat upon exposure to light; and heating the adhesion polymer chains include subjecting the nanoparticles to light. Non-limiting examples of the nanoparticles include gold nanoparticles. In these embodiments, the adhesion polymer chain is a thermo-responsive polymer. Non-limiting examples of thermo-responsive polymers include poly(N-isopropylacrylamide) (PNIPAM) and its copolymers. In certain embodiments, the heat-promoted cross-linking of the adhesion polymer chains is reversible, and the method further includes cooling the adhesion polymeric network to break the cross-links between the adhesion polymer chains.

In still other specific embodiments, joining two or more of the adhesion polymer chains together by a bonding force include forming covalent bonds between the adhesion polymer chains. In certain embodiments, the covalent bonds between the adhesion polymer chains include an ester bond, an amide bond, an O—C bond, a N—C bond, a Si—O bond or a combination thereof. In these embodiments, the adjacent two adhesion polymer chains may have two reacting groups suitable for forming these covalent bonds. For instance, the adjacent two adhesion polymer chains may each have an OH group and a COOH group, respectively, to form an ester bond; the adjacent two adhesion polymer chains may each have a NH₂ (or NHR, where R is an alkyl group) and a COOH group, respectively, to form an amide bond; the adjacent two adhesion polymer chains may each have an O and a C-LG group, respectively, to form an O—C bond (where LG refers to a good leaving group such as Cl, Br, I, OMs, or OTs); and the adjacent two adhesion polymer chains may each have a NH₂ (or NHR, where R is an alkyl group) and a C-LG group (where LG refers to a good leaving group such as Cl, Br, I, OMs, or OTs), respectively, to form a N—C bond.

In some embodiments, the formed covalent bond is easily broken (and thus the bonding force resulting from the covalent bond is reversible). Non-limiting examples of covalent bonds easily broken include an ester bond, which can be broken, e.g., upon hydrolysis of the ester bond. In other embodiments, the formed covalent bond is stable and thus the bonding force resulting from the covalent bond may be permanent. Non-limiting examples of such stable covalent bonds include a N—C bond and an O—C bond.

Therefore, in some embodiments, the topological adhesion of the first and second materials achieved by the method described herein together is reversible: the bond or force joining the adhesion polymer chains to form the adhesion polymeric network may be broken to dissemble the adhesion polymeric network, thereby dissociating the first and second materials from each other.

### Examples

In some embodiments, an approach to molecularly stitch wet materials is described. Each wet material, to be called an adherend, has a pre-existing polymer network. The molecular stitch uses polymer chains to form a new polymer network, in response to a trigger. This new polymer network is localized at the interface between the two adherends, and in topological entanglement with the network of the adherend on either side. It is through this topological entanglement that the new polymer network stitches the two pre-existing polymer networks. To debond, the topologically entangled networks must disentangle—that is, at least one of the three networks must break. In some embodiments, the adhesion polymer chain is also referred to as the stitching polymers and the adhesion polymeric network is also referred to as the stitching polymer network. In these embodiments, the topological adhesion is also referred to as topohesion for brevity and the stitching polymers is referred to as the topohesive. The stitching polymer network functions as a molecular suture.

The design is twofold. First, polymer chains can be triggered to form a network, localized at the interface of two adherends, and in topological entanglement with the pre-existing networks of the adherends. Second, the stitching polymer network can be flexible enough to retain the softness of the adherends, and yet strong enough to achieve adhesion energy comparable to the bulk toughness of the adherends by eliciting the hysteresis in the adherends, without requiring any functional groups from the adherends. Functional groups may exist in tissues and hydrogels for other reasons, but need not form any bonds with the stitching polymers.

Topological entanglement has played fundamental roles in polymers. It has in recent decades led to hydrogels and elastomers of exceptional modulus, strength, and toughness. Topological entanglement has also been used to achieve adhesion by diffusing monomers into adherends and polymerizing in-situ. Such a bonding method starts with monomers, and often involves invasive and toxic chemical reactions, as well as ultraviolet irradiation. No attempt has been reported to trigger polymer chains to form a stitching network and achieve strong adhesion between hydrogels and tissues.

Topological adhesion was illustrated by using pH as a trigger. This pH-triggered topological adhesion mimics the formation of strong byssal threads by a mussel (Mytilus californianus Conrad 1837). When a foot of the mussel attaches to a surface, the distal depression of the foot secretes an aqueous solution of proteins at pH˜3. When the foot lifts off, the surrounding seawater of pH˜8 flows in, and the proteins form a strong network.

To formulate a topohesive, polymer chains that dissolve in water in one pH range and form a polymer network in another pH range were used. An aqueous solution of the stitching polymer chains at one value of pH was prepared. The two adherends have another value of pH, representative of that in a physiological environment. The solution of the stitching polymer chains was placed between the two adherends. In response to the pH in the adherends, the polymer chains form a third network. Adhesion energy above 1,000 Jm⁻² was readily achieved when the stitching polymer network elicits hysteresis in the wet materials. By choosing different species of stitching polymers, strong adhesion in full range of pH was achieved. Hydrogels were bonded to various porcine tissues (liver, heart, artery, skin, and stomach). Furthermore, the molecular suture is removable, on-demand, by changing the pH back to the soluble range of the polymer chains.

In these embodiments, two pieces of hydrogels using chitosan chains were bonded first. Hydrogels are relatively simple and well-characterized systems, and structurally similar to living tissues and extensively used in medicine. Chitosan chains are biopolymers widely used in bioengineering. The amine groups on chitosan are responsive to changes in pH. The dissociation [NH₃⁺] [symbol] [NH₂]+[H⁺] has the equilibrium constant Ka=[NH₂][H⁺]/[NH₃⁺]. By definition pKa=−log[Ka] and pH=−log [H⁺], so that log[NH₂]−log[NH₃⁺]=pH−pKa. For chitosan, pKa=6.5. When pH<6.5, [NH₂]<[NH₃⁺], and the chitosan chains dissolve in water as a polyelectrolyte (FIG. 1a). When pH>6.5, [NH₂]>[NH₃⁺], and the NH₂—OH hydrogen bond promotes the chitosan chains to form a network (FIG. 1b).

An aqueous solution of chitosan of pH=5 was prepared and the solution was placed between two hydrogels of pH=7 (FIG. 1c). The chitosan chains diffused into the two hydrogels and, in response to the pH in the hydrogels, formed a third network (FIG. 1d). The chitosan network is in topological entanglement with the networks of the two hydrogels. Whether such a chitosan network will form depends on two concurrent kinetic processes: the diffusion of chitosan chains into the hydrogel, and the formation of the chitosan network.

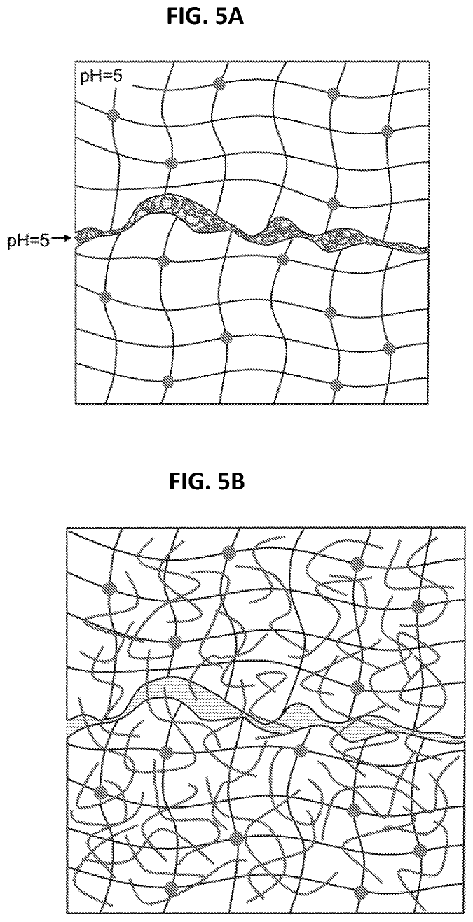

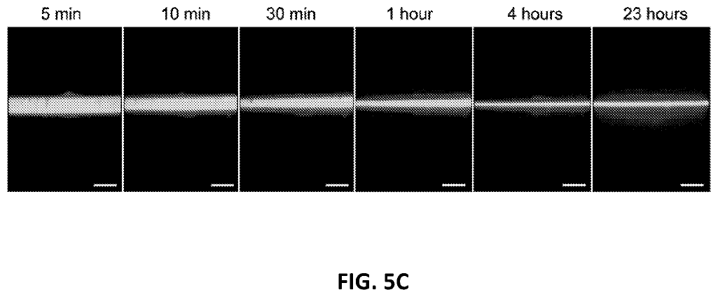

To confirm the formation of the chitosan network localized at the interface, the chitosan chains were labeled with fluorescein isothiocyanate (FITC), and their diffusion was tracked using confocal microscopy (FIG. 1E). In the first hour, the chitosan chains diffused away from the interface, shown by the gradual thinning of the chitosan-rich layer and the thickening of the diffusion layer. However, fewer chitosan chains diffused away in the next few hours, and finally chitosan chains ceased to diffuse at 24 hours. As a comparison, when the same chitosan solution was placed between two hydrogels of pH=5, the chitosan chains kept diffusing into the hydrogels, and no chitosan network formed (FIGS. 5A-5C).

According to the Rouse model, the diffusivity of a chitosan chain in water is D=kT/(Nηb), where kT is the temperature in the unit of energy, η is the viscosity of water, b is the size of the repeating unit of the chitosan chain, and Nis the number of the repeating units. Taking kT=10⁻²¹ J, η=10⁻³ Pa·s, b=10⁻⁹ m, and N=1,000, D˜10⁻¹² m²s⁻¹ was obtained, which is much lower than the diffusivity of H⁺ and OH⁻ (˜10⁻⁹ m²s⁻¹). The time scale is estimated as h²/D˜hours, where h is the diffused chitosan thickness (˜100 μm from the confocal image). This estimate roughly agrees with the experimental observation.

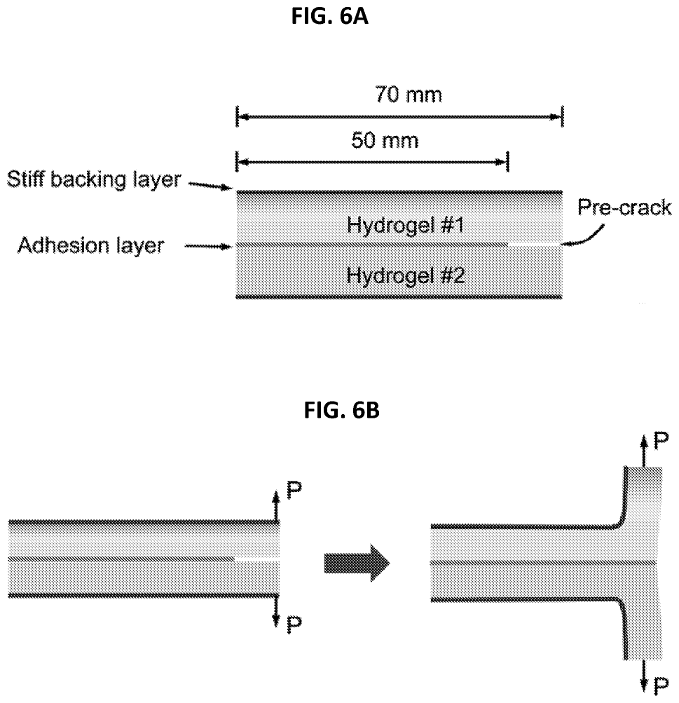

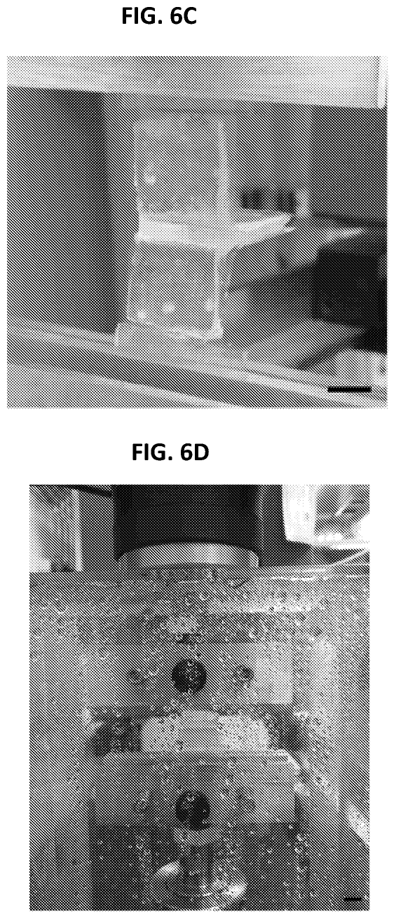

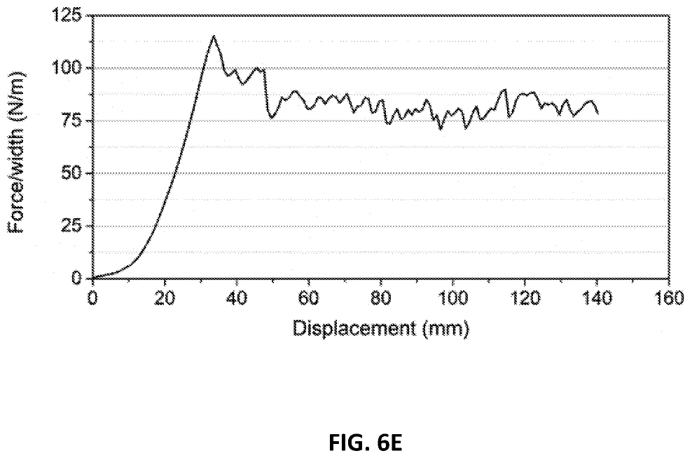

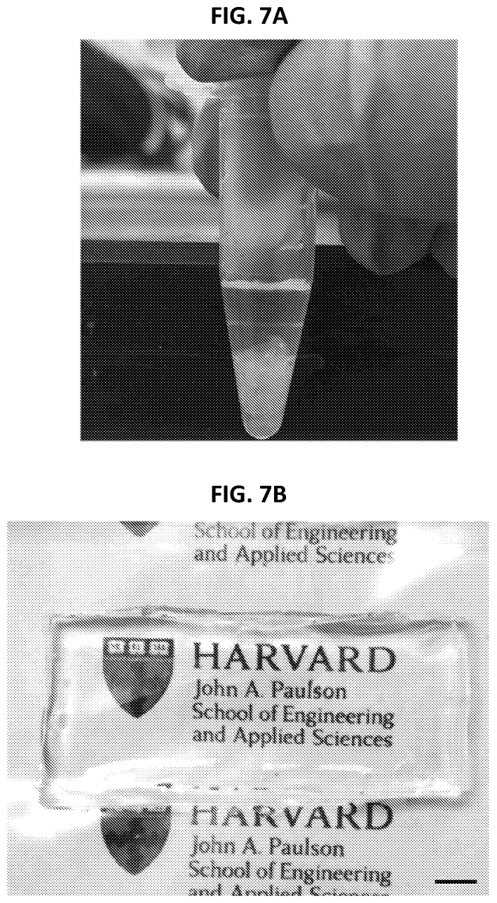

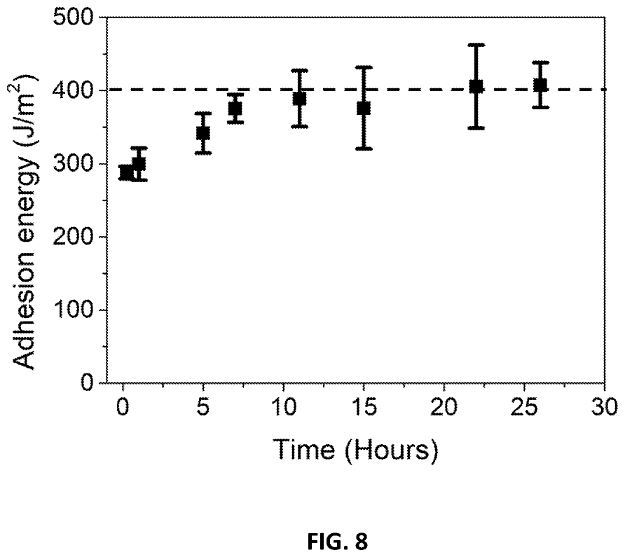

The formation of chitosan network in topological entanglement with the networks of the hydrogels by measuring the adhesion energy was ascertained. The dissociation energy of a hydrogen bond is weak (˜10 kT) compared to that of a covalent bond (˜140 kT). However, tens to hundreds of hydrogen bonds in aggregation result in a high bonding energy. The network of pure chitosan is still fragile, but the chitosan network in topological entanglement with the network of the adherends achieves adhesion energy comparable to the bulk toughness of the adherends (FIG. 1F, Table 1). For example, the bulk toughness of PDMA is ˜22 Jm⁻² and the adhesion energy is ˜33 Jm⁻², and the bulk toughness of alg-PAAM is ˜8,000 Jm⁻² and the adhesion energy is ˜2,000 Jm⁻². Moreover, the bonded hydrogels are stretchable and transparent (FIGS. 6a-6d and 7A-7B). Chitosan chains to stitch PAAM hydrogel to various porcine tissues were also used: liver, heart, artery, and skin (FIG. 1G). The skin exhibits a relatively high toughness among the soft organs (˜1,000 Jm⁻²), thus the corresponding adhesion energy is also high (˜100 Jm⁻²).

TABLE 1
Non-Limiting Examples of Hydrogels.

| | | Charges on | Interaction |
|---|---|---|---|
| Polymer chain of hydrogel | Molecular structure | polymer chains | with chitosan |
| Poly(hydroxyethylmethamylate) (PHEMA) | 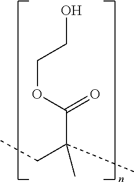 | Neutral | Weak hydrogen bond |
| Poly(acrylamide) (PAAM) | 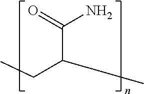 | Neutral | Weak hydrogen bond |
| Poly(dimethylacrylamide) (PDMA) | 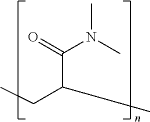 | Neutral | Weak hydrogen bond |
| Poly(N-isopropylacrylamide) (PNIPAM) | 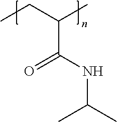 | Neutral | Weak hydrogen bond |
| Sodium polyacrylate (NaPAA) | 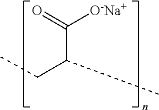 | Negatively charged | Weak hydrogen bond and ionic bond |
| [2-(Acryloyloxy)ethyl] trimethylammonium chloride (PDMAEA) | 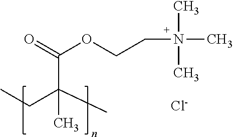 | Positively charged | Weak hydrogen bond and positive charge repulsion |
| Polyacrylamide/alginate | 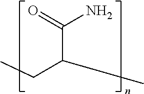 | Neutral/negatively charged | Weak hydrogen bond and ionic bond |
| | 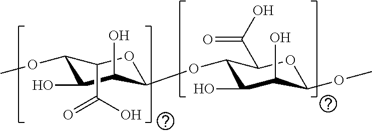 | | |

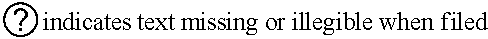

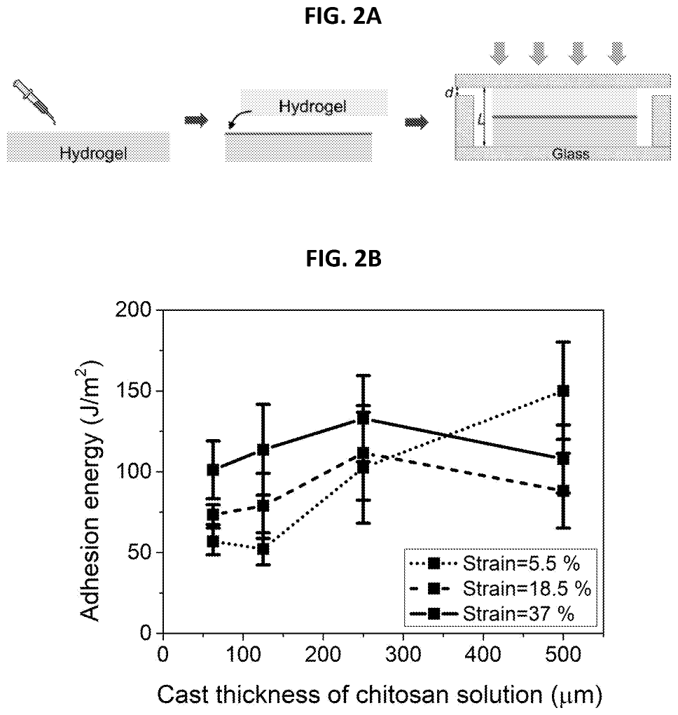

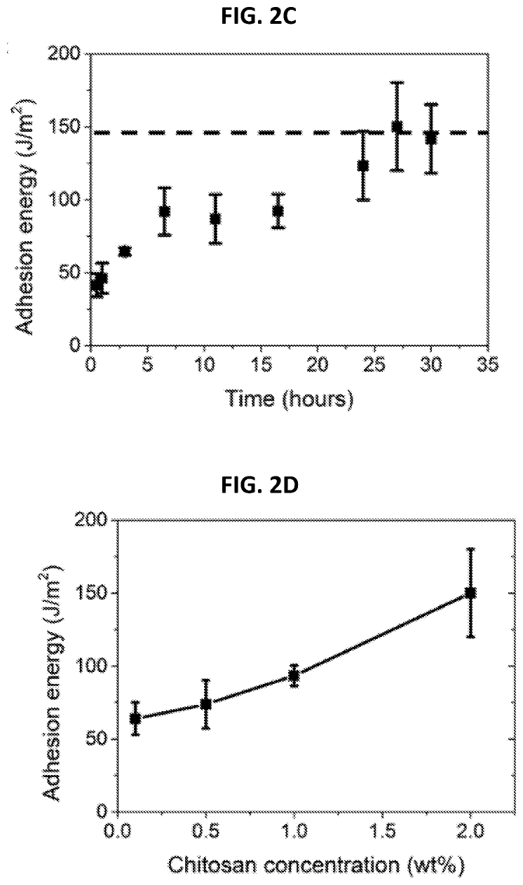

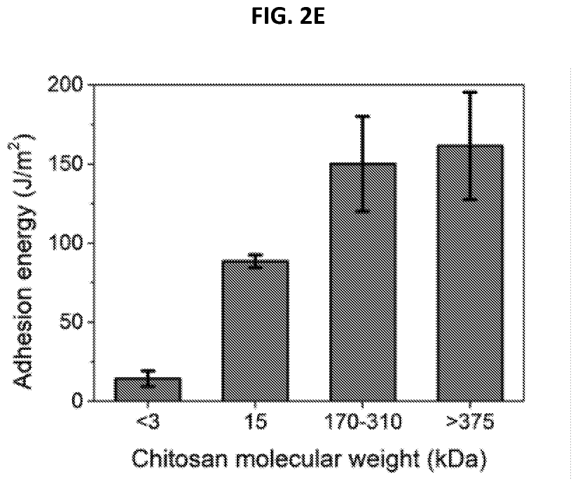

The procedure for preparing adhesion can greatly affect the adhesion energy. To illustrates some of the effects, a chitosan solution was spread on one piece of PAAM hydrogel, another piece of PAAM hydrogel placed on top, and the two hydrogels compressed with a strain d/L (FIG. 2A). The maximum adhesion was achieved at the combination of a solution of 500 μm thickness and a strain of 5.5%, and the thinner chitosan layer and larger strain lead to weaker adhesion energy (FIG. 2B). The change of adhesion energy was monitored over time, and it was found that the adhesion energy established to ˜50 Jm⁻² within 30 min, and then approached an equilibrium value of ˜150 Jm⁻² after 24 hours (FIG. 2C). The slow kinetics may be associated with the slow formation of the chitosan network. Similar phenomena have been observed in the aging of PAAM-PVA hydrogels and PAAM-chitosan hydrogels, as well as in the slow recovery of alg-PAAM hydrogels. For topological adhesion, the kinetics of adhesion depend on stitching polymers. For example, poly(4-aminostyrene) (PAS) reached adhesion energy of ˜300 Jm⁻² within the first 15 min, and saturated to ˜400 Jm⁻² after 10 hours (FIG. 8). The controlled kinetics of adhesion may find clinical advantages, as the initial relatively small adhesion is often sufficient to hold tissues or hydrogels, but allows repositioning. In addition, the chitosan chains need to be sufficiently concentrated and long to ensure strong adhesion (FIGS. 2D-2E).

The fluids in human tissues vary greatly in pH. The blood, the cerebrospinal fluid, and the cellular fluid of muscle and skin are nearly neutral (pH=6-7.4), the pancreatic fluid and bile are alkaline (pH=7.6-8.8), and the gastric fluids are extremely acidic (pH=1-3.5). Next, several species of stitching polymers were used to achieve strong adhesion in full range of pH. In addition to chitosan, which forms a network when pH>6.5, three other species of stitching polymer chains were used: PAS forms a network when pH>4.6, alginate forms a network when pH<3.5, and cellulose forms a network when pH<13 (FIG. 3A, Table 2). The four species of polymers were used to bond PAAM hydrogels of various values of pH, and confirmed that adhesion was established only when the pH of the hydrogels was in the network-forming range of each species of the stitching polymers (FIGS. 3B-3E).

TABLE 2
Non-Limiting Examples of Stitching Polymers.

| Stitching | Molecular | | Soluble |
|---|---|---|---|
| polymers | structure | pKa | pH |
| Poly(4- aminestyrene) | 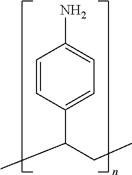 | 4.6 | <4.6 |
| Chitosan | 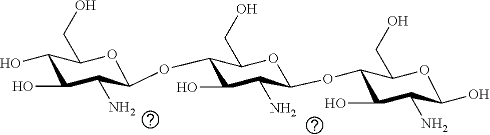 | 6.5 | <6.5 |
| Alginate | 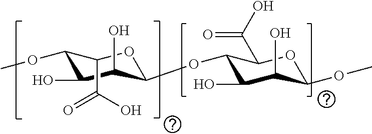 | 3.5 | >3.5 |
| Cellulose | 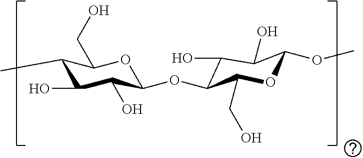 | 13 | >13 |
| Stitching | | Bonding | |
| polymers | Soluble state | pH | Crosslinks in networks |
| Poly(4- aminestyrene) | 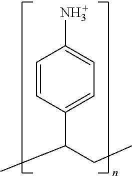 | >4.6 | NH₂—NH₂H-bond π-π stacking |
| Chitosan | 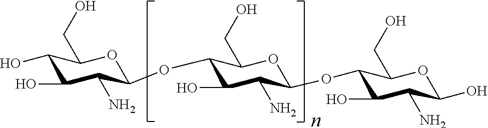 | >6.5 | NH₂—OH H-bond |
| Alginate | 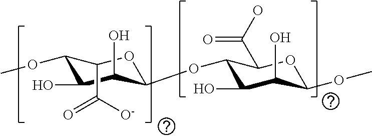 | <3.5 | COOH—COOH H-bond |
| Cellulose | 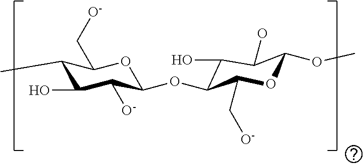 | <13 | OH—OH H-bond |

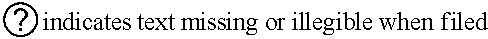

In the network-forming range of pH, the adhesion energy is low when the pH of hydrogel is either close to, or far from, the pKa of the stitching polymers, and exhibits a maximum in the middle. This finding was interpreted using chitosan as an example. When the pH of hydrogel is close to pKa of chitosan, the positively charged amine NH₃⁺ and neutral amine NH₂ are comparable in numbers. This may lead to insufficient number of hydrogen bonds and thus a weak chitosan network. When the pH of hydrogel far exceeds pKa, the chitosan chains at interface are neutralized to form network much faster, which impedes the diffusion of chitosan chains into both hydrogels. This may lead to insufficient topological entanglements with both hydrogel networks.

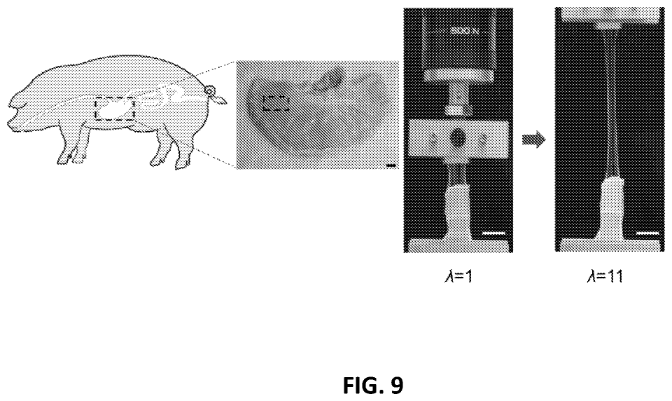

Topological adhesion enables the design of adhesives for extreme pH environment. The strong adhesion between a hydrogel and a porcine stomach tissue in an extremely low pH (˜1.5) with cellulose solution (FIG. 9) was demonstrated. The low pH resembles the physiological environment inside the stomach. The bonding by lap shear test was tested. The hydrogel used is much softer than the stomach tissue. The lap-shear test showed that the hydrogel remained firmly bonded to the tissue over a stretch of 11 times of its initial length.

The molecular stitch is removable, on-demand, when the pH is changed back to the soluble range of the stitching polymers. This capability was demonstrated using chitosan-stitched PAAM hydrogels. The top hydrogel was fixed to a rigid acrylic plate and hung the bottom hydrogel with a weight. The weight itself did not cause debonding. Water was then dripped at the bonding front for several times. No debonding was observed after 12 trails of dripping (FIG. 3F). In comparison, hydrochloric acid (1 M) was dripped at the same bonding front. The debonding progressively advanced at every single dripping, until two hydrogels completely detached (FIG. 3G). The capability of on-demand removal of the molecular stitch may motivate future design of smart adhesives or bandages.

Topological adhesion involves three polymer networks: the pre-existing networks of the two adherends, and the newly formed network of the stitching polymers. To disentangle, at least one of the three networks must break. The intrinsic energy to break a network is 10-100 Jm⁻². This picture is fundamentally different from polymer chains physically entangled with the networks of two adherends without forming a network. The polymer chains can be disentangled and pulled out without breaking any network, requiring an energy of ˜1 Jm⁻².

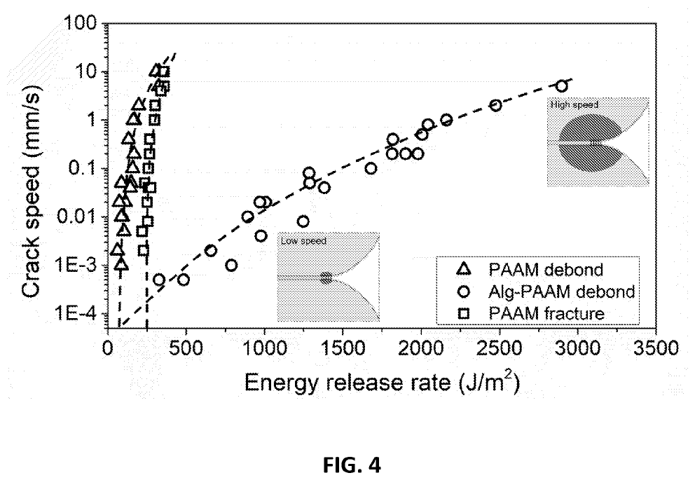

To confirm the formation of a strong and stable stitching polymer network, the speed of crack was measured as a function of energy release rate. When a crack extends at a certain speed, the measured energy release rate results from two processes: the disentanglement at the crack front and the hysteresis in the adherends. The latter effect reduces when the crack speed is low. The energy release rate was measured at crack speeds across many orders of magnitude (FIG. 4). For chitosan-stitched PAAM hydrogels, the energy release rate arrives at a constant value of ˜60 Jm⁻² as the crack speed approaches zero. This relatively high value supports that the chitosan network and the PAAM network are topologically entangled: the debond breaks at least one of the networks.

To find whether the chitosan network or the PAAM network breaks, the slow-crack test was conducted for a homogeneous PAAM hydrogel. The energy release rate of the homogeneous PAAM hydrogel is higher than that of the chitosan-stitched PAAM hydrogels at all crack speeds, and arrives at a constant value of ˜250 Jm⁻² as the crack speed approaches zero. This comparison indicates that the chitosan-stitched PAAM hydrogels disentangled by the breaking of the chitosan network, not the PAAM network. The slow-crack experiment by itself, however, is unable to differentiate whether the chitosan network broke by the scission of the chitosan chains, or by the unzipping of the hydrogen bonds between the chitosan chains.

Topological adhesion confirms a fundamental principle in fracture mechanics: adhesion is strong if debond elicits hysteresis in the adherends. The slow-crack experiment was conducted for chitosan-stitched hybrid alg-PAAM hydrogels. The stress-stretch curve of an alg-PAAM hydrogel exhibits pronounced hysteresis. The slow-crack data showed that the chitosan suture is strong enough to elicit the hysteresis in the alg-PAAM to achieve strong adhesion. However, the stress-strain curve of the alg-PAAM hydrogel is rate-dependent. The energy release rate is ˜3000 Jm⁻² at a crack speed of 10 mms⁻¹, but reduces to ˜400 Jm⁻² at 1 μms⁻¹. By extrapolation, the energy release rate approaches 60 Jm⁻² as the crack speed approaches zero. The hysteresis in the bulk greatly amplifies the energy release rate at high crack speed, but contributes negligibly to the energy release rate at low crack speed.

In summary, these experiments taken together show that suitable polymers form a network in topological entanglement with the networks of two wet materials, and the topologically entangled networks lead to strong adhesion without requiring any functional groups from the wet materials. A given species of stitching polymers works for any wet materials upon triggering by an environmental stimulus, so long as the wet materials do not prevent the formation of the stitching polymer network. The topological adhesion can be applied to a large area, so long as the stitching polymers cover the entire bonding area. The stitching polymer network functions as a suture in the molecular scale, and this molecular suture can be designed to be permanent, transient, or removable on-demand. Topological adhesion is general, which is not limited to be triggered by pH but can be potentially triggered by other stimuli such as salt, temperature, and light, along with their corresponding responsive polymers. For example, PNIPAM can be used as the thermo-responsive polymer, which forms a network when the temperature is above the lower critical solution temperature. As another example, when gold nanoparticles are mixed into the wet materials, they generate heat upon exposure to light, which trigger the topological adhesion using thermo-responsive polymers. It is hoped that the topological adhesion opens a field of development to achieve strong adhesion between wet materials, while retaining softness.

### Materials

All chemicals were purchased and used without further purification. Monomers for hydrogels included acrylamide (AAm; Sigma-Aldrich, A8887), 2-Hydroxyethyl methacrylate (HEMA; Sigma-Aldrich, 128635), N-isopropylacrylamide (NIPAM; Sigma-Aldrich, 415324), N,N-Dimethylacrylamide (DMA; Sigma-Aldrich, 274135), acrylic acid (AAc; Sigma-Aldrich, 147230) and [2-(Acryloyloxy)ethyl]trimethylammonium chloride solution (DMAEA; Sigma-Aldrich, 496146). To prepare alg-polyacrylamide tough hydrogels, ionically crosslinkable alginate biopolymer (FMC Biopolymer, Manugel GMB) was used and crosslinked with calcium sulfate slurry (calcium sulfate dihydrate; Sigma-Aldrich, c3771). N,N′-methylenebisacrylamide (MBAA; Sigma-Aldrich, M7279) was used as the covalent crosslinker. Ammonium persulfate (APS; Sigma-Aldrich, A9164), sodium persulfate (NaPS, Sigma-Aldrich, 216232) and α-Ketoglutaric acid (Sigma-Aldrich, 75890) were used as initiators for polymerization in different pH. N,N,N′,N′-tetramethylethylenediamine (TEMED; Sigma-Aldrich, T7024) was used as crosslinking accelerator for APS and NaPS.

The stitching polymers employed in the study include chitosan chains of four different molecular weights: Mw>375,000 Da (Sigma-Aldrich, 419419), Mw˜190,000-310,000 Da (Sigma-Aldrich, 448877), Mw˜15,000 Da (Polysciences, 21161-50) and Mw≤3,000 Da (Carbosynth, OC28900), alginic acid sodium salt (Mw˜120,000-190,000 Da, Sigma-Aldrich, 180947), poly(4-aminostyrene) (PAS, Mw>150,000 Da; Polysciences, 02823-1) and cellulose (Mw˜500,000 Da; Sigma-Aldrich, 435236).

### Preparation of Hydrogels

Polyacrylamide hydrogel: 40.56 g acrylamide powder was first dissolved in 300 ml deionized water, and MBAA was added as covalent crosslinker (MBAA to acrylamide weight ratio is 0.0006:1). To prepare PAAM hydrogel of pH<7, α-Ketoglutaric acid was used as UV initiator (α-Ketoglutaric acid to acrylamide weight ratio is 0.002:1). The pH of precursor solution was tuned by dripping HCl solution. The precursor solution was subsequently poured into a glass mold and covered with a 3-mm-thick glass plate, and exposed under UV irradiation (30 W, 365 nm curing UV light, McMaster-Carr) for one hour and set for hours to complete polymerization. To prepare PAAM hydrogel of pH≥7, APS or NaPS was used as initiator (APS to acrylamide weight ratio is 0.01:1; NaPS to acrylamide weight ratio is 0.007:1), in coupling with TEMED as crosslinking accelerator (TEMED to acrylamide weight ratio is 0.0028:1). NaOH was dripped to achieve a desired pH. The precursor solution was then poured into a glass mold and covered with a 3-mm-thick glass plate to complete polymerization.

PHEW hydrogel: 46 ml HEMA was dissolved in 200 ml DI water. MBAA (MBAA to HEMA weight ratio is 0.00033:1), TEMED (TEMED to HEMA weight ratio is 0.002:1), and APS (APS to HEMA weight ratio is 0.0054:1) was sequentially added into the HEMA solution and mixed. The precursor solution was then poured into a glass mold and covered with a 3-mm-thick glass plate to complete polymerization.

PNIPAAM hydrogel: 21.52 g NIPAM powder was dissolved in 100 ml DI water. MBAA (MBAA to NIPAM weight ratio is 0.000377:1), TEMED (TEMED to NIPAM weight ratio is 0.0023:1), and APS (APS to NIPAM weight ratio is 0.006:1) was sequentially added into the NIPAM solution and mixed. The precursor solution was then poured into a glass mold and covered with a 3-mm-thick glass plate to complete polymerization.

PDMA hydrogel: 4.12 ml DMA was diluted in 20 ml DI water. 0.0031 g MBAA (MBAA to DMA weight ratio is 0.00078:1), TEMED (TEMED to DMA weight ratio is 0.003:1), and APS (APS to DMA weight ratio is 0.0067:1). The precursor solution was mixed and poured into a glass mold and covered with a 3-mm-thick glass plate to complete polymerization.

NaPAA hydrogel: 8.22 ml acrylic acid (AAc) was dissolved in 21.78 ml DI water. 0.004864 g MBAA (MBAA to AAc weight ratio is 0.00056:1) and 0.009 g α-Ketoglutaric acid (α-Ketoglutaric acid to AAc weight ratio is 0.001:1) was sequentially added. NaOH was added to tune the pH of the solution to be neutral. The precursor solution was mixed and poured into a glass mold and covered with a 3-mm-thick glass plate, exposed under UV irradiation for one hour, and set for hours to complete polymerization.

PDMAEA-Q hydrogel: 16 ml DMAEA was dissolved in 14 ml DI water. NaOH was added to tune the pH of the solution to be neutral. MBAA (MBAA to DMAEA weight ratio is 0.0017:1) and APS (APS to DMAEA weight ratio is 0.0027:1) were sequentially added. The precursor solution was then poured into a glass mold and covered with a 3-mm-thick glass plate to complete polymerization.

Alg-PAAM tough hydrogel: 40.56 g acrylamide powder and 6.78 g alginate powder were dissolved together in 300 ml deionized water. MBAA (MBAA to acrylamide weight ratio is 0.0006:1) and TEMED (TEMED to acrylamide weight ratio is 0.0028:1) were then sequentially added. The solution was mixed and degassed. Next, APS (APS to acrylamide weight ratio is 0.01:1) was added as initiator and calcium sulfate slurry as ionic crosslinker (CaSO4 to acrylamide weight ratio is 0.022:1) was added into the solution. To prevent fast gelation of alginate, the precursor solution was quickly mixed and immediately poured into a glass mold and covered with a 3-mm-thick glass plate to complete polymerization.

### Preparation of Stitching Polymer Solutions

Chitosan solution: 4-Morpholineethanesulfonic acid (MES hydrate; Sigma-Aldrich, M8250) was used to prepare an acidic buffer solution. The MES buffer solution was first prepared by dissolving 0.976 g MES hydrate powder into 50 ml DI water and adjusted the pH to 4.5 by dripping NaOH with a pH meter (Mettler Toledo SevenEasy™ Series Meters). 1 g chitosan powder (Mw˜190,000-310,000 Da) was then added into the buffer solution and sufficiently stirred with a magnetic stirring bar until chitosan powder was completely dissolved. The final pH of solution was about 5.

PAS solution: The MES buffer solution was prepared as described above and adjusted the pH to 1 by dripping HCl with a pH meter. 1 wt % PAS was then added into the buffer solution and sonicated in an ultrasonic bath (Branson Ultrasonics) with a constant temperature of 48° C. overnight. After PAS was completely dissolved, the solution was clear with a deep yellow color. The pH of solution was re-adjusted back to 4.

Alginate solution: 2 wt % alginic acid sodium salt was dissolved in DI water. The solution was vigorously mixed and sonicated in an ultrasonic bath with a constant temperature of 48° C. for an hour.

Cellulose solution: The cellulose solution was prepared following the recipe described in J. Cai, L. Zhang, Macromol. Biosci. 2005, 5, 539. Briefly, 7 wt % NaOH pellets (Macron) and 12 wt % urea powders (Sigma-Aldrich, U5128) were directly dissolved in DI water. The alkaline solution was pre-cooled at −20° C. before use. Next, 2 wt % cellulose powders were added into the alkaline solution and vigorously mixed until transparent solution was yielded.

### Experimental Procedure of Bonding

The prepared chitosan solution was directly spread on the surface of one piece of hydrogel with thickness of ˜500 Another piece of hydrogel was placed on top, and the two pieces of hydrogels compressed with 5.5% strain with customized glass molds. The whole structure was then sealed in a plastic bag to prevent dehydration. The weight of bilayer was measured after 24 hours and showed only 0.2 wt % difference, indicating negligible water loss during the procedure.

### Confocal Microscopy

FITC labeled chitosan (Chitosan-Fluorescein; Akina, Inc., KITO-9) was used to track the diffusion of chitosan chains in hydrogels. PAAM hydrogels were prepared with pH of 5, 7, and 12. A 2 wt % chitosan solution was prepared, with FITC-chitosan and chitosan (Mw˜190,000-310,000 Da) in a weight ratio of 1:9. The solution was covered from light. Two pieces of polyacrylamide hydrogels were bonded with the FITC-chitosan solution. The entire sample was imaged with a confocal fluorescence microscope (Leica tcs-sp5), with an excitation wavelength of 490 nm and emission wavelength of 525 nm for FITC. A series of confocal images were taken by scanning the sample along its thickness at different time after bonding. The images were reconstructed in 3D using ImageJ to visualize the diffusion kinetics and the adhesion layer.

### T-Peeling Tests for Measuring the Adhesion Energy

All tests except the low-peeling-rate tests were conducted in an open air and at room temperature. For tests with low peeling rate (<0.04 mm s⁻¹), the samples were tested in a humidity chamber to prevent dehydration of hydrogel. The humidity chamber was home-made with acrylic sheets and equipped with a household cool mist humidifier and a humidity control system (Zoo med, HygroTherm Humidity and Temperature Controller). The relative humidity in the chamber was maintained about 92%. The hydrogel sample was 50 mm in length, 20 mm in width, and 1.5 mm in thickness (FIGS. 6a-6e). The back sides of the hydrogels were glued with stiff polyester films of thickness 100 μm (clear polyester film, McMaster-Carr) with Krazy glue (cyanoacrylate). For hydrogels with pH near neutral, the Krazy glue was directly used to bond the polyester film and the hydrogel. However, for hydrogels with pH too low or too high, the Krazy glue failed to bond the polyester film and hydrogel. The low pH inhibited the polymerization of cyanoacrylate, and the high pH made the polymerization too fast. Both led to unsatisfactory bonding quality. To overcome this issue, a few drops of NaOH (1 M) or HCl (1 M) solution were dripped on the back surface of the hydrogel to adjust the local pH close to neutral. Consequently, the Krazy glue can be successfully applied to bond the polyester film and the hydrogel. The volume of the few drops applied was much smaller than that of the hydrogel. The T-peeling test was conducted immediately following the dripping. The time was only several minutes, insufficient for the OH⁻ or H⁺ ions to diffuse across the whole hydrogel to neutralize it. As a result, the pH was only changed locally on the back surface of the hydrogel, but did not affect the pH in the bulk hydrogel, especially close to the interface between the two pieces of hydrogels, where topological adhesion was performed. The polyester films restrict deformation of hydrogels during the T-peeling test. The free ends of the hydrogels were fixed to an Instron testing machine with 10 N or 500 N load cells. The peeling rate was fixed at 0.4 mm s⁻¹. For slow crack tests, the peeling rate was varied by orders of magnitude. The peeling force as a function of displacement was recorded. The adhesion energy was calculated as twice the value of the peeling force at plateau divided by the width of the sample.

### Pure Shear Tests for Measuring Bulk Toughness of Hydrogels

Pure shear tests were used to measure the fracture energy of the hydrogels. For each test, two samples of the same hydrogel with dimension 50×10×1.5 mm (90×10×1.5 mm for PAAM hydrogels) were made: one with a pre-crack of 20 mm cut by razor blade, and the other without. All the samples were tested individually on an Instron testing machine with a 500 N load cell. The loading rate was fixed at 0.2 mm s⁻¹. The unnotched samples were used to measure the stress-stretch curve. In the reference state when the sample is not deformed, the height of the sample is H; after loading, the height becomes λH. The area beneath the stress-stretch curve gives the strain energy density stored in the hydrogel, W(λ). The notched samples were used to measure the critical stretch λc, where the notch turns into a running crack. The fracture energy is calculated as Γ=W(λc) H. R. Rivlin, A. G. Thomas, J. Polym. Sci., Part A: Polym. Chem. 1953, 10, 291.

### Bonding a Hydrogel and a Stomach Tissue

A fresh porcine stomach was obtained from a local grocery store. A piece of stomach tissue was carefully cut with a size of 3.7 cm×4.4 cm, and subsequently soaked in a simulated gastric acid (Ricca Chemical, 7108-32, simulated Gastric Fluid (without Pepsin), 0.2% (w/v) sodium chloride in 0.7% (v/v) hydrochloric Acid, pH-1.5) for one hour. Cellulose solution was then spread on the tissue surface and immediately attached a piece of PAAM hydrogel under gentle pressure. The PAAM hydrogel is much softer than the stomach tissue. The elastic modulus of the PAAM hydrogel is about 1 kPa, while the elastic modulus of the stomach tissue is about 1 MPa. See C. T. McKee, J. A. Last, P. Russell, C. J. Murphy, Tissue Engineering Part B: Reviews, 2011, 17, 155. The bonding by the lap shear test was tested after several hours.

### On-Demand Detachment

Chitosan solution was used to bond two pieces of PAAM hydrogels. The top hydrogel was attached to a rigid acrylic plate, and the bottom hydrogel was attached to a weight of 100 g (equivalent to an energy release rate of 50 J m⁻²). The weight itself does not cause debonding. Next, water was dripped directly at the bonding front, and observed no debonding after 12 trails. Hydrochloride solution (1 M) was then dripped directly at the same bonding front, and observed progressive debonding after every single dripping, until the two pieces of hydrogels completely detached.

It will be appreciated that while one or more particular materials or steps have been shown and described for purposes of explanation, the materials or steps may be varied in certain respects, or materials or steps may be combined, while still obtaining the desired outcome. Additionally, modifications to the disclosed embodiment and the invention as claimed are possible and within the scope of this disclosed invention.

1. A composite material comprising:
a first material comprising a first polymeric network;
a second material comprising a second polymeric network; and
an adhesion polymeric network comprising a plurality of adhesion polymer chains joined together by a bonding force and interwoven with the first and second polymeric networks to adhere the first and second materials together,
wherein the adhesion polymeric network is not covalently bonded to the first or second polymeric network.

2. The composite material of claim 1, wherein at least one of the first and second polymeric network is cross-linked.

3. The composite material of claim 1, wherein at least one of the first and second material is a dry material or a wet material comprising a solvent.

4. The composite material of claim 3, wherein the solvent is water.

5. The composite material of claim 3, wherein the solvent is an organic solvent.

6. The composite material of claim 1, wherein each of the first and second materials is independently selected from the group consisting of a hydrogel, a tissue, and an elastomer.

7. The composite material of claim 1, wherein the first and second polymeric networks each independently comprises one or more polymers selected from the group consisting of poly(hydroxyethylmethacrylate) (PHEMA), poly(acrylamide) (PAAM), poly(dimethylacrylamide) (PDMA), poly(N-isopropylacrylamide) (PNIPAM), sodium polyacrylate (NaPAA), [2-(Acryloyloxy)ethyl] trimethylammonium chloride (PDMAEA), polyacrylamide, alginate, and a combination thereof.

8. The composite material of claim 1, wherein the adhesion polymer chains comprise one or more bio-compatible polymers.

9. The composite material of claim 1, wherein each of the adhesion polymer chains is independently selected from the group consisting of poly(4-aminestyrene), chitosan, alginate, cellulose, poly(N-isopropylacrylamide), polymers containing silane groups and/or catechol groups, a copolymer thereof, a terpolymer thereof, and a block copolymer thereof.

10. The composite material of claim 1, wherein the bonding force results from a bond or interaction selected from the group consisting of hydrogen bond, ionic bond, van der Waals interaction, covalent bond, π-π stacking, cation-π-π interaction, host-guest interaction, and a combination thereof.

11. The composite material of claim 1, wherein the bonding force results from a bond or interaction which is permanent, transient, or reversible.

12. The composite material of claim 11, wherein the bond or interaction is reversible.

13. The composite material of claim 12, wherein the bond is a hydrogen bond, a covalent bond or an ionic bond.

14. The composite material of claim 13, wherein each of the adhesion polymer chains is independently selected from the group consisting of poly(4-aminestyrene), chitosan, alginate, cellulose, poly(N-isopropylacrylamide), polymers containing silane groups and/or catechol groups a copolymer thereof, a terpolymer thereof, and a block copolymer thereof.

15. The composite material of claim 1, wherein the bonding force results from a bond or interaction which is formed in response to a stimulus.

16. The composite material of claim 15, wherein the stimulus is selected from the group consisting of pH, salt, temperature, light, and a combination thereof.

17. The composite material of claim 1, wherein the first and second materials are adhered with an adhesion energy of more than about 10, 50, 100, 200, 300, 500, 600, or 1000 Jm⁻².

18-59. (canceled)
